(19) United States
(12) Patent Application Publication
(10) Pub. No.: US 2018/0193350 A1
(43) Pub. Date: Jul. 12, 2018

(54) TREATMENT OF NUT MIDLINE CARCINOMA

(71) Applicant: Tensha Therapeutics, Inc., South San Francisco, CA (US)

(72) Inventors: Steven B. Landau, Wellesley, MA (US); Michael H. Kagey, Arlington, MA (US)

(21) Appl. No.: 15/739,905

(22) PCT Filed: Jun. 24, 2016
(86) PCT No.: PCT/US2016/039270

§ 371 Date: Dec. 26, 2017

Related U.S. Application Data
(60) Provisional application No. 62/185,203, filed on Jun. 26, 2015.

(51) Int. Cl. A61K 31/551; A61P 35/00; G01N 33/574

(52) U.S. Cl. CPC A61K 31/551; A61P 35/00; G01N 2333/70553; G01N 2800/52; G01N 33/57407

(57) ABSTRACT

Disclosed herein is a method of treating nuclear protein in testis (NUT) midline carcinoma (NMC) in a subject in need thereof, comprising administering an effective amount of a bromodomain inhibitor, wherein the effective amount can be determined according to the expression levels of CD11b, which monitors responsiveness of the NMC to the bromodomain inhibitor. Also disclosed herein is a method of determining a bromodomain inhibitor treatment regimen in a subject suffering from NMC.

### RELATED APPLICATION

This application claims the benefit of U.S. Provisional Application No. 62/185,203, filed on Jun. 26, 2015. The entire teachings of the above application are incorporated herein by reference.

### BACKGROUND OF THE INVENTION

NUT midline carcinoma (or NMC) is a rare form of cancer characterized by a chromosomal rearrangement in which a portion of the NUT (nuclear protein in testis) gene on chromosome 15 is fused to a BRD (bromodomain protein) gene or other, as yet unidentified, gene (French, et al., Cancer Res. 63(2):304-307 (2003); French, et al., J. Clin. Oncol. 22(20):4135-4139 (2004); French, et al., Oncogene 27(15):2237-42 (2008)). NUT fusion genes encode oncoproteins that maintain cells in an undifferentiated state and promote their rapid and uncontrolled growth.

For the majority of cases, the translocation occurs between NUT and BRD3 or BRD4, leading to a fusion protein consisting of the bromodomains and virtually the entire coding sequence of NUT (French et al., Ann. Rev. Pathol. 7:247-265, (2012)). Mechanistically, BRD-NUT appears to block differentiation of the cancer cells in part by decreasing global histone acetylation levels through the sequestration of the histone acetyl transferase p300 in subnuclear foci French, et al., Oncogene 27:2237-42 (2008); Schwartz, et al., Cancer Res. 71:2686-96, (2011)). Furthermorne, the BRD4-NUT fusion protein binds to the promoter of the MYC oncogene and activates expression, contributing to the undifferentiated proliferative state of NMC cells (Grayson, et al., Oncogene 33:1736-42 (2014). The frequent involvement of midline structures in the head, neck, mediastinal, and other midline structures, suggest that NMCs arise from primitive neural crest-derived cells. NMCs are very aggressive clinically, respond poorly to conventional chemotherapy, and are almost uniformly fatal. Even with aggressive surgery, radiation therapy, and systemic chemotherapy, the median lifespan is only 6.7 months (French, et al., Head Neck Pathol. (2013)). NMC can occur in children and adults of all ages.

Accordingly, there is a significant unmet need for therapies with increased efficacy in treating NMC. The present application provides such therapies.

### SUMMARY OF THE INVENTION

The present invention relates to a method of nuclear protein in testis (NUT) midline carcinoma (NMC) therapy in a subject in need of treatment, comprising administering an effective amount of an inhibitor of the bromodomain and extra terminal (BET) family of bromodomains. In particular, the methods provided herein are based, in part, on the identification of CD11b expression level on cells (e.g., monocytes) as an indicator of disease responsiveness (or disease activity) to the BET inhibitor.

In one aspect, the present invention provides a method of treating a patient suffering from nuclear protein in testis (NUT) midline carcinoma (NMC), comprising: administering an effective amount of a bromodomain inhibitor to the patient in a current cycle of a treatment regimen having multiple cycles, each cycle including an on-drug and an off-drug segment, wherein the patient exhibits a CD11b expression reduction of less than about 50% relative to a baseline level, wherein the CD11b expression is measured during the current cycle or a prior cycle.

In another aspect, the invention provides a method of monitoring a treatment response in a patient suffering from nuclear protein in testis (NUT) midline carcinoma (NMC), comprising: a) administering a predetermined amount of a bromodomain inhibitor to the patient using a treatment regimen having multiple cycles, each cycle comprising an on-drug and an off-drug segment; and b) quantifying a CD11b expression level in a sample collected from the patient; wherein a CD11 b expression reduction of about 50% or more relative to a baseline level indicates a positive response to the treatment regimen.

In other aspects, the invention also provides a method of determining a treatment regimen in a patient suffering from nuclear protein in testis (NUT) midline carcinoma (NMC), comprising: a) administering a predetermined amount of a bromodomain inhibitor to the patient in a first cycle of a treatment regimen having multiple cycles, each cycle including an on-drug and an off-drug segment; b) quantifying a CD11b expression level in a sample collected from the patient during the first cycle; and c) determining whether to modify the first cycle or a subsequent cycle of the treatment regimen, wherein a CD11b expression reduction of less than about 50% relative to a baseline level indicates that the first cycle or the subsequent cycle should be modified, thereby determining the treatment regimen in a patient suffering from NMC.

Many cell lines of solid tumor origin, including NMC, are sensitive to bromodomain inhibitors (e.g., TEN-010). Notably, the present invention reveals a relationship between CD11b levels and responsiveness to bromodomain inhibitor therapy that is specific to NMC patients. Thus, CD11b expression levels on cells (e.g. monocytes) can be used to monitor responsiveness to a BET inhibitor (e.g., TEN-010) in NMC patients, and to enable modification of a pre-existing BET inhibitor therapy to enhance efficacy of NMC treatment. The ability to monitor and modify an ongoing bromodomain therapy regimen for NMC treatment is particularly desirable given the highly aggressive nature of the disease.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views.

FIG. 1 shows CD11b levels in patients undergoing treatment with TEN-010. The designation “004-001 (NMC)” indicates the patient as one who is suffering from NMC. “MESF” refers to Molecules of Equivalent Soluble Fluorochrome. Measurements taken at the indicated time points are denoted as “C#D#” wherein C# refers to the cycle number, and D# refers to the number of days in the indicated cycle. For example, C2D1 refers to cycle 2, day 1.

FIGS. 2A-2F illustrate a comparison of lactate dehydrogenase (LDH) levels and CD11b levels in each patient presented in FIG. 1 undergoing TEN-010 treatment, wherein LDH levels are represented on the left y-axis, and CD11b levels are represented on the right y-axis. “MESF” refers to Molecules of Equivalent Soluble Fluorochrome. Measurements taken at the indicated time points are denoted as “C#D#” wherein C# refers to the cycle number, and D# refers to the number of days in the indicated cycle. For example, C4D22 refers to cycle 4, day 22.

### DETAILED DESCRIPTION OF THE INVENTION

A description of example embodiments of the invention follows.

A bromodomain is an approximately 110 amino acid protein domain that recognizes monoacetylated lysine residues such as those on the N-terminal tails of histones. Acetylation of lysine residues is a post-translational modification with broad relevance to cellular signalling and disease biology. Enzymes that ‘write’ (histone acetyltransferases, HATs) and ‘erase’ (histone deacetylases, HDACs) acetylation sites are an area of extensive research in current drug development, but very few potent inhibitors that modulate the ‘reading process’ mediated by acetyl lysines have been described. The principal readers of ε-N-acetyl lysine (Kac) marks are bromodomains (BRDs), a diverse family of evolutionary conserved protein-interaction modules. Proteins that contain BRDs have been implicated in the development of a large variety of diseases. Targeting BRD-mediated protein-protein interaction has emerged as a promising avenue for drug development for the large number of diseases that are caused by aberrant acetylation of lysine residues.

The BET inhibitor class of compounds targets and inhibits the bromodomain and extra terminal (BET) family of proteins. The BET family currently consists of four proteins, the ubiquitously expressed BRD2, BRD3 and BRD4, and the testis specific BRDT (Jones et al., Genomics 45:529-34 (1997); Paillisson et al., Genomics 89:215-23 (2007)). BET proteins are transcription cofactors that are involved in regulating cell-cycle progression, proliferation, energy homeostasis, spermatogenesis and inflammatory responses (Belkina and Denis, Nat. Rev. Cancer 12:465-77, (2012); Matzuk et al., Cell 150:673-84, (2012); Nicodeme et al., Nature 468:1119-23, (2010); Wang et al., Biochem. J. 425:71-83, (2010); Wu and Chiang, JBC 282:13141-45, (2007). Each family member contains two amino-terminal tandem bromodomains and a conserved extra-terminal (ET) domain that is also involved in protein-protein interactions (Rahman et al., Mol. Cell Biol. 31:2641-52, (2011). BET proteins regulate gene expression by binding acetylated chromatin at promoters and enhancers (see, e.g., Draker et al., PLoS Genet 8, e1003047, (2012)). BET proteins stimulate gene expression by recruiting positive transcription elongation factor b (P-TEFb) (see, e.g., Zhang et al., JBC 287:43137-55, (2012)). P-TEFb promotes the release of RNA polymerase II from promoters, resulting in productive transcriptional elongation and active gene expression. JQ1 (referred to herein as S-JQ1S), a known BET inhibitor, specifically binds the bromodomains of the BET family (Bres et al., Curr. Opin. Cell Biol. 20:334-340, (2008)).

The specific BET family member BRD4 has been directly implicated in regulating cell-cycle progression. BRD4 is a bookmarking factor that remains bound to chromosomes during mitosis and recruits P-TEFb to genes to promote activation of an early G1 transcriptional program (Dey et al., MBC 20:4899-4909, (2009); Yang et al., MBC 28:967-76, (2008)). Decreasing BRD4 protein levels results in the failure of expression of key G1 growth associated genes as the cell exits mitosis, leading to a G1 arrest and apoptosis (Dey et al., MBC 20:4899-4909, (2009); Yang et al., MBC 28:967-76, (2008); Mochizuki et al., JBC 283:9040-48, (2008)). Similar results have been obtained with JQ1 (i.e., JQ1S as described herein) treatment (a known BET inhibitor), which displaces BRD4 from mitotic chromosomes and significantly delays the activation of early G1 genes (Zhao et al., Nat. Cell Biol. 13:1295-1304, (2011)).

BRD3 and BRD4 are also implicated in NMC, which predominantly results from a translocation between the NUT gene and BRD3 and BRD4. NMC occurs in the midline, most commonly in the head, neck, or mediastinum, as poorly differentiated carcinomas with variable degrees of squamous differentiation. This tumor is defined by rearrangement of the “nuclear protein in testis” (NUT) gene on chromosome 15q14. In most cases, NUT is involved in a balanced translocation with the BRD4 gene on chromosome 19p13.1, an event that creates a BRD4-NETT fusion gene. Variant rearrangements, some involving the BRD3 gene, occur in the remaining cases. NMC may be diagnosed by detection of NUT rearrangement by fluorescence in situ hybridization, karyotype analysis, or RT-PCR. Due to its rarity and lack of characteristic histologic features, most cases of NMC currently go unrecognized.

NMC is defined herein as any malignant epithelial tumor with rearrangement of the NUT gene. In approximately ⅔ of cases, NUT (chromosome 15q14) is fused to BRD4, on chromosome 19p13.1, forming the BRD4-NUT fusion gene. In the remaining ⅓ of cases, the partner gene is BRD3 or other uncharacterized gene. These are referred to as NUT-variant fusion genes. The histologic features of NMC are not distinctive, and diagnosis is based on detection of the NUT rearrangement. NUT rearrangements define NMCs, and for this reason the diagnosis is never in question once rearrangement of NUT has been demonstrated. Methods of detecting such rearrangements are known and available in the art. Through implication of BRD3 and BRD4 in NMC, BET bromodomain inhibitors also have promise as a targeted therapy for NMC (Filippakopoulos et al., Nature 468:1067-73, (2010)).

Methods of BET Inhibitor Therapy in NUT Midline Carcinoma (NMC)

The present invention is based, in part, on the identification of CD11b expression level on cells (e.g., monocytes) as an indicator of NMC responsiveness (or disease activity) to a BET inhibitor. CD11b (also known as integrin α_{M}) is an integrin family member which pairs with CD18 (also known as integrin β₂) to form the CR3 complement heterodimer receptor (also known as Macrophage-1 antigen, Mac-1, integrin α_{M}β₂, or macrophage integrin). CD11b is expressed on the surface of leukocytes including monocytes, neutrophils, natural killer cells, granulocytes and macrophages, as well as on some spleen cells and bone marrow cells. Functionally, CD11b regulates leukocyte adhesion and migration to mediate the inflammatory response.

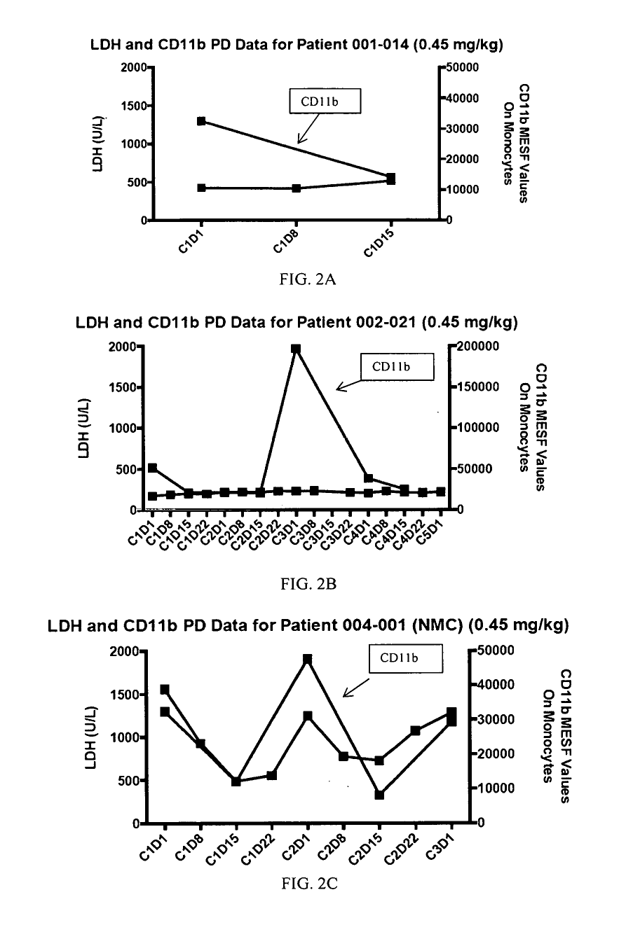

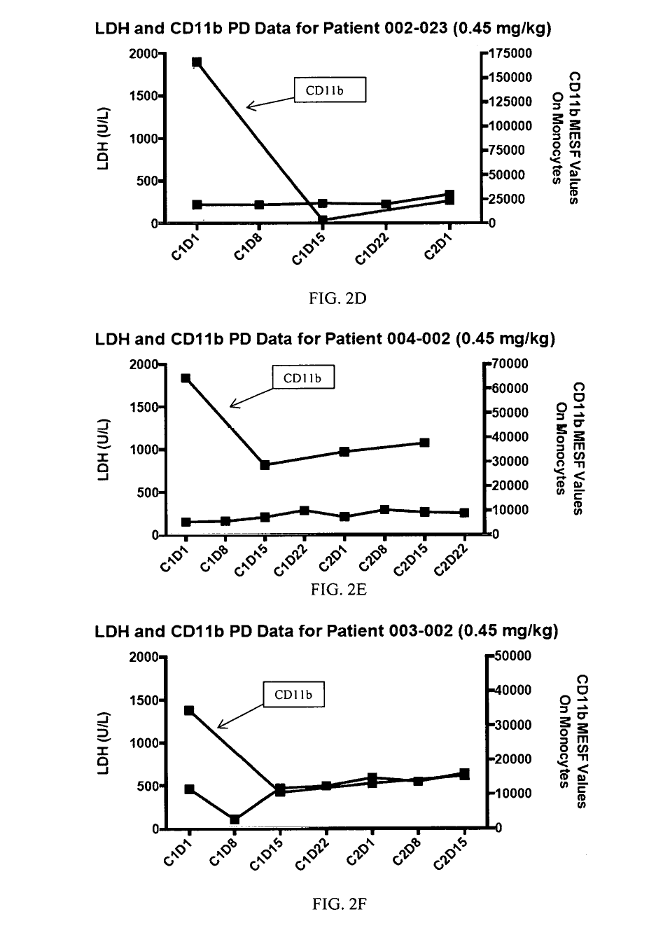

As exemplified herein, CD11b levels can be used to monitor responsiveness to a bromodomain inhibitor therapy in a patient suffering from NMC, as validated by lactate dehydrogenase (LDH) levels, which is a known clinical marker of cancer progression. Briefly, the present invention demonstrates that, in an NMC patient, CD11b expression levels tracked closely with LDH levels throughout the course of TEN-010 therapy (FIG. 2C). In contrast, CD11b expression levels are independent of LDH levels in non-NMC patients (FIGS. 2A, 2B, and 2D-2F, in particular 2B). Thus, while not wishing to be bound by any theory, monitoring CD11b levels on monocytes enables one to follow NMC disease activity in a patient undergoing a bromodomain inhibitor therapy. As described herein, CD11b levels can be measured to determine whether an NMC patient will require more or less bromodomain inhibitor in subsequent cycle(s) of treatment, or whether an NMC patient will require an earlier or later commencement of a subsequent cycle of bromodomain inhibitor treatment, or any combination thereof.

Accordingly, in one aspect, the present invention provides a method of treating a patient suffering from nuclear protein in testis (NUT) midline carcinoma (NMC), comprising: administering an effective amount of a bromodomain inhibitor to the patient in a current cycle of a treatment regimen having multiple cycles, each cycle including an on-drug and an off-drug segment, wherein the patient exhibits a CD11b expression reduction of less than about 50% relative to a baseline level, wherein the CD11b expression is measured during the current cycle or a prior cycle.

As used herein “treating” includes any evidence of antitumor activity including, but not limited to, delaying or preventing the progression of clinical indications related to the NMC. For example, disease progression can be slowed. Additional, evidence of antitumor activity includes reduction in tumor growth, or prevention of further growth or reduction in tumor metabolic activity, as detected by standard imaging methods known in the art, including, for example, computed tomography (CT) scan, magnetic resonance imaging (MRI), chest x-ray, and CT/positron emission tomography (CT/PET) scans, and evaluated according to guidelines and methods known in the art. For example, responses to treatment can be evaluated through the Response Evaluation Criteria in Solid Tumors (RECIST) (Revised RECIST Guideline version 1.1; see Eisenhauer et al., Eur. J. Cancer 45(2):228-47, 2009). Thus, in some embodiments, “treating” refers to a Complete Response (CR), which is defined according to the RECIST guideline as the disappearance of all target lesions, or a Partial Response (PR), which is defined as at least a 30% decrease in the sum of diameter of target lesions, taking as reference the baseline sum diameters. Other means for evaluating tumor response to treatment include evaluation of tumor markers and evaluation of performance status (e.g., assessment of creatinine clearance; see Cockcroft and Gault, Nephron. 16:31-41, 1976). Response evaluation for lymphoma patients is based upon Lugano Classification.

The terms “bromodomain inhibitor” and “BET inhibitor” are used interchangebly. Both terms refer to a class of compounds that targets and inhibits the bromodomain and extra terminal (BET) family of proteins. Examples of bromodomain inhibitors are described in detail herein. In one embodiment, the bromodomain inhibitor is TEN-010.

As used herein, the term “patient” refers to a mammal, preferably a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The term “effective amount” as used herein refers to an effective dosage over a specified treatment cycle within a treatment regimen that includes multiple cycles, each cycle comprising on-drug and off-drug segments, such that the effect of the treatment regimen achieves and maintains a CD11b expression level during any cycle that is at least 50% reduced as compared to baseline levels of CD11b (i.e., 50% or more reduction in CD11b compared to a baseline level). In certain embodiments, the effect of the treatment regimen achieves and maintains 60%, 70%, 80%, or 90% or more reduction in CD11b compared to a baseline level.

As used herein, a “cycle” within a treatment regimen refers to a specified period of time (e.g., number of days) that consists of “on-drug” and “off-drug” segments, wherein “on-drug” refers to a period of time during which drug is administered, whereas “off-drug” refers to a period of time during which no drug is administered. In one embodiment, a cycle consists of one on-drug segment and one off-drug segment. In another embodiment, a cycle can consist of one continuous on-drug segment with no off-drug segment (e.g., continuous dosing), wherein the cycle is still defined as having a specified number of days (e.g., 28 days). In this scenario, the delineation of one cycle from the next cycle is determined by the number of specified days (e.g., 28 days); a subsequent cycle can be designed to have the same, higher, or lower dose of bromodomain inhibitor as compared to a prior cycle, as determined according to the methods of the invention.

As used herein, a “current” cycle refers to the cycle presently ongoing.

As used herein, a “prior” cycle refers to any prior cycle within a treatment regimen, including a cycle that occurred one cycle prior to the current cycle, as well as a cycle that occurred more than one cycle prior to the current cycle.

A cycle can consist of a number of days deemed appropriate by a skilled medical professional, and will vary depending on the nature of the disease, the dose of the drug being administered, the health of the patient, the intended result, and the like. By way of example, a cycle of a bromodomain inhibitor treatment regimen for treating NMC can be about 15 to about 35 days. In one embodiment, a cycle can be about 28 days, having 21 on-drug days, and 7 off-drug days. As will be appreciated by those of skill in the art, a cycle having any combination of the number of “on” and “off” drug days (including zero off-drug days) can be designed as deemed appropriate by a skilled medical professional.

A patient's sample can be obtained and the CD11b expression level measured during any portion of a segment (on or off) of a cycle for comparison against a baseline level to determine and/or administer an effective amount of a bromodomain inhibitor during the current cycle. For example, the CD11b expression level can be measured during the off-drug segment of a prior cycle. If, by way of example, the CD11b expression level during any portion of the off-drug segment of the prior cycle is reduced by less than about 50% relative to a baseline level (i.e., CD11b level is higher than desired and treatment is not effective), then a higher dose of bromodomain inhibitor can be administered in the current cycle. Alternatively, or in addition, the number of days in the off-drug segment of the prior cycle can be shortened (relative to a pre-determined number of days in the off-segment of a cycle) to begin the current cycle earlier. In contrast, if it is determined that the CD11b expression level is favorable (i.e., treatment is effective), then the bromodomain inhibitor dose can be maintained or decreased.

As another example, if the CD11b expression level during the on-drug segment of the current cycle is reduced by less than about 50% relative to a baseline level, then a higher dose of bromodomain inhibitor can be administered in the ongoing current cycle. In this second example, it is also possible to increase the number of days in the on-drug segment of the current cycle in addition to, or alternatively to, increasing the dose of bromodomain inhibitor.

As used herein, the “baseline” level refers to the level of CD11b expression measured in an NMC patient prior to receiving the first dose of treatment (at pre-dose).

In certain embodiments, the sample obtained from the patient is a blood sample.

In other aspects, the present invention also provides a method of determining a treatment regimen in a patient suffering from NMC, comprising: a) administering a predetermined amount of a bromodomain inhibitor to the patient in a first cycle of a treatment regimen having multiple cycles, each cycle including an on-drug and an off-drug segment; b) quantifying a CD11b expression level in a sample collected from the patient during the first cycle; and c) determining whether to modify the first cycle or a subsequent cycle of the treatment regimen, wherein a CD11b expression reduction of less than about 50% relative to a baseline level indicates that the first cycle or the subsequent cycle should be modified, thereby determining the treatment regimen in a patient suffering from NMC.

As used herein, a “predetermined amount” refers to an amount of a bromodomain inhibitor determined for a patient based, for example, on criteria previously determined, but that which is potentially currently not effective due to, for example, a change in disease status.

As used herein, a “first cycle” refers to a current, ongoing cycle of treatment, and does not necessarily refer to the actual first cycle of a bromodomain inhibitor treatment regimen.

In certain embodiments, the first cycle or the subsequent cycle is modified by increasing the length of the on-drug segment, decreasing the length of the off-drug segment, increasing the predetermined amount of the bromodomain inhibitor, or a combination thereof. The table below summarizes some examples of possible scenarios and modifications to a treatment regimen, when it is determined that CD1 lb expression reduction is less than about 50% relative to a baseline level (i.e., CD11b level is higher than desired and disease responsiveness is not at a suitable level). If it is determined that CD11b expression reduction is favorable (i.e., disease responsiveness is at a suitable level), then it can be desirable to, e.g., decrease the bromodomain inhibitor dose, or delay the commencement of the next cycle, or both.

TABLE 1
Possible modifications to bromodomain treatment regimen

| When CD11b | Possible modifications if CD11b expression reduction |
|---|---|
| measured | is less than about 50% relative to baseline level |
| On-drug segment | increase the number of days in the on-drug segment |
| of current cycle | of the current cycle |
| | increase the dose of bromodomain inhibitor during the |
| | on-drug segment of the current cycle |
| | increase the dose of bromodomain inhibitor in the |
| | subsequent (e.g. next) cycle |
| | shorten number of days in the off-drug segment of the |
| | current cycle |
| | any combination of above |
| | if a cycle consists of only an on-drug segment with no |
| | off-drug segment (e.g. continuous dosing), then |
| | increase the dose of bromodomain inhibitor in the |
| | current cycle, or the next cycle of “on-drug only” |
| | cycle |
| Off-drug segment | shorten number of days in current off-drug segment |
| of current cycle | (i.e., commence subsequent cycle earlier) |
| | increase number of days on-drug segment of |
| | subsequent cycle |
| | increase dose of bromodomain inhibitor in subsequent |
| | cycle |
| | shorten off-drug segment of subsequent cycle |
| | any combination of above |

CD11b expression levels on cells (e.g., monocytes) can be quantified using a variety of methods known and available in the art. In one example, CD11b expression levels on monocytes can be quantitued by flow cytometry.

In another aspect, the present invention provides a method of monitoring a treatment response in a patient suffering from NMC, comprising: a) administering a predetermined amount of a bromodomain inhibitor to the patient using a treatment regimen having multiple cycles, each cycle comprising an on-drug and an off-drug segment; and b) quantifying a CD11b expression level in a sample collected from the patient; wherein a CD11b expression reduction of about 50% or more relative to a baseline level indicates a positive response to the treatment regimen.

BET Inhibitors

Definitions

“Alkyl” means an optionally substituted saturated aliphatic branched or straight-chain monovalent hydrocarbon radical having the specified number of carbon atoms. Thus, “(C₁-C₆) alkyl” means a radical having from 1-6 carbon atoms in a linear or branched arrangement. “(C₁-C₆)alkyl” includes methyl, ethyl, propyl, iso-propyl (or i-propyl), butyl, sec-butyl, tert-butyl, pentyl, hexyl and the like. The terms “alkyl”, “alkoxy”, “hydroxyalkyl”, “haloalkyl”, “aralkyl”, “alkoxyalkyl”, “alkylamine”, “dialkylamine”, “alkylamino”, “dialkylamino”, “alkoxycarbonyl”and the like, used alone or as part of a larger moiety includes both straight and branched saturated chains containing one to twelve carbon atoms.

“Alkylene” means an optionally substituted saturated aliphatic branched or straight-chain divalent hydrocarbon radical having the specified number of carbon atoms. Thus, “(C₁-C₆)alkylene” means a divalent saturated aliphatic radical having from 1-6 carbon atoms in a linear arrangement, e.g., —[(CH₂)ₙ]—, where n is an integer from 1 to 6, “(C₁-C₆)alkylene” includes methylene, ethylene, propylene, butylene, pentylene and hexylene. Alternatively, “(C₁-C₆)alkylene” means a divalent saturated radical having from 1-6 carbon atoms in a branched arrangement, for example: —[(CH₂CH₂CH₂CH₂CH(CH₃)]—, —[(CH₂CH₂CH₂CH₂C(CH₃)₂]—, —[(CH₂C(CH₃)₂CH(CH₃))]—, and the like. A specific branched C₃-alkylene is

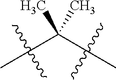

and a specific C₄-alkylene is

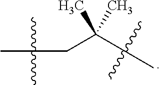

“Alkenyl” means branched or straight-chain monovalent hydrocarbon radical containing at least one double bond and having specified number of carbon atoms. Alkenyl may be mono or polyunsaturated, and may exist in the E or Z onfiguration. For example, “(C₂-C₆)alkenyl” means a radical having from 2-6 carbon atoms in a linear or branched arrangement.

“Alkynyl” means branched or straight-chain monovalent hydrocarbon radical containing at least one triple bond and having specified number of carbon atoms. For example, “(C₂-C₆)alkynyl” means a radical having from 2-6 carbon atoms in a linear or branched arrangement.

Each alkyl or alkylene in Structural Formulas depicted below can be optionally and independently substituted with one or more substituents.

“Aryl” or “aromatic” means an aromatic monocyclic or polycyclic (e.g. bicyclic or tricyclic) carbon-containing ring system. In one embodiment, “aryl” is a 6-12 membered monocyclic or bicyclic system. Aryl systems include, but are not limited to, phenyl, naphthalenyl, fluorenyl, indenyl, azulenyl, and anthracenyl.

“Cycloalkyl” means a saturated aliphatic cyclic hydrocarbon ring. “Cycloalkyl” includes 3- to 12-membered saturated aliphatic cyclic hydrocarbon rings. Thus, “(C₃-C₇)cycloalkyl” means a hydrocarbon radical of a 3- to 7-membered saturated aliphatic cyclic hydrocarbon ring. A (C₃-C₇)cycloalkyl includes, but is not limited to cyclopropyl, cyclobutyl, cyclopentyl, cyclohexyl and cycloheptyl.

A cycloalkyl moiety can be monocyclic, fused bicyclic, bridged bicyclic, spiro bicyclic, or polycyclic. For example, monocyclic (C₃-C₈)cycloalkyl means a radical having from 3 to 8 carbon atoms arranged in a monocyclic ring. Monocyclic (C₃-C₈)cycloalkyl includes, but is not limited to, cyclopropyl, cyclobutyl, cyclopentyl, cyclohexyl, cycloheptyl and cyclooctane.

Monocyclic ring systems have a single ring structure. They include saturated or unsaturated aliphatic cyclic hydrocarbon rings (e.g., cycloalkyl, cycloalkenyl, or cycloalkynyl) or aromatic hydrocarbon rings (e.g., aryl) having the specified number of carbon atoms. The monocyclic ring system can optionally contain 1 to 5 heteroatoms in the ring structure wherein each heteroatom is independently selected from the group consisting O, N and S (e.g., heterocycloalkyl, heterocycloalkenyl, heterocycloalkynyl or heteroaryl). When the heteroatom is N, it can be optionally substituted with alkyl, cycloalkyl, alkylene-cycloalkyl, heterocycloalkyl, alkylene-heterocycloalkyl, aryl, alkylene-aryl, heteroaryl, alkylene-heteroaryl, each of which can be optionally substituted with one or more halogen, ═O, hydroxy, alkoxy, haloalkyl, alkyl, etc. When the heteroatom is S, it can be optionally mono- or di-oxygenated (i.e., —S(O)— or —S(O)₂—). Examples of monocyclic ring systems include, but are not limited to, cyclopropyl, cyclobutyl, cyclopentyl, cyclohexyl, cycloheptyl, cyclooctane, azetidine, pyrrolidine, piperidine, piperazine, azepane hexahydropyrimidine, tetrahydrofuran, tetrahydropyran, oxepane, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine, tetrahydro-2H-1,2-thiazine 1,1-dioxide, and isothiazolidine 1,1-dioxide, tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

Bicyclic ring systems have two rings that have at least one ring atom in common. Bicyclic ring systems include fused, bridged and spiro ring systems. The two rings can both be aliphatic (e.g., cycloalkyl, cycloalkene, cycloalkyne, or heterocycloalkyl), both be aromatic (e.g., aryl or heteroaryl), or a combination thereof. The bicyclic ring systems can optionally contain 1 to 5 heteroatoms in the ring structure wherein each heteroatom is independently selected from the group consisting O, N and S. When the heteroatom is N, it can be substituted with H, alkyl, cycloalkyl, alkylene-cycloalkyl, heterocycloalkyl, alkylene-heterocycloalkyl, aryl, alkylene-aryl, heteroaryl, alkylene-heteroaryl, each of which can be optionally substituted with one or more halogen, ═O, hydroxy, alkoxy, haloalkyl, alkyl, etc. When the heteroatom is S, it can be optionally mono- or di-oxygenated (i.e. —S(O)— or —S(O)₂—).

A fused bicyclic ring system has two rings which have two adjacent ring atoms in common. The two rings can both be aliphatic (e.g., cycloalkyl, cycloalkene, cycloalkyne, or heterocycloalkyl), both be aromatic (e.g., aryl or heteroaryl), or a combination thereof. For example, the first ring can be cycloalkyl or heterocycloalkyl, and the second ring can be a cycloalkyl, cycloalkene, cycloalkyne, aryl, heteroaryl or a heterocycloalkyl. For example, the second ring can be a (C₃-C₆)cycloalkyl, such as cyclopropyl, cyclobutyl, cyclopentyl and cyclohexyl. Alternatively, the second ring can be an aryl ring (e.g., phenyl). Examples of fused bicyclic ring systems include, but are not limited to, 6,7,8,9-tetrahydro-5H-benzo[7]annulene, 2,3-dihydro-1H-indene, octahydro-1H-indene, tetrahydronaphthalene, decahydronaphthalene, indoline, isoindoline, 2,3-dihydro-1H-benzo[d]imidazole, 2,3-dihydrobenzo[d]oxazole, 2,3-dihydrobenzo[d]thiazole, octahydrobenzo[d]oxazole, octahydro-1H-benzo[d]imidazole, octahydrobenzo[d]thiazole, octahydrocyclopenta[c]pyrrole, 3-azabicyclo[3.1.0]hexane, 3-azabicyclo[3.2.0]heptane, 5,6,7,8-tetrahydroquinoline and 5,6,7,8-tetrahydroisoquinoline, and 2,3,4,5-tetrahydrobenzo[b]oxepine.

A Spiro bicyclic ring system has two rings which have only one ring atom in common. The two rings can both be aliphatic (e.g., cycloalkyl, cycloalkene, cycloalkyne, or heterocycloalkyl), both be aromatic (e.g., aryl or heteroaryl), or a combination thereof. For example, the first ring can be a cycloalkyl or a heterocycloalkyl and the second ring can be a cycloalkyl, a cycloalkene, a cycloalkyne, an aryl, a heteroaryl, or a heterocycloalkyl. Examples of spiral bicyclic ring systems include, but are not limited to, spiro[2.2]pentane, spiro[2.3]hexane, spiro[3.3]heptane, spiro[2.4]heptane, spiro[3.4]octane, spiro[2.5]octane, azaspiro[4.4]nonane, 7-azaspiro[4.4]nonane, azaspiro[4.5]decane, 8-azaspiro[4.5]decane, azaspiro[5.5]undecane, 3-azaspiro[5.5]undecane, and 3,9-diazaspiro[5.5]undecane.

A bridged bicyclic ring system has two rings which have three or more adjacent ring atoms in common. The two rings can both be aliphatic (e.g., cycloalkyl, cycloalkene, cycloalkyne, or heterocycloalkyl), both be aromatic (e.g., aryl or heteroaryl), or a combination thereof. For example, the first ring can be a cycloalkyl or a heterocycloalkyl and the other ring is a cycloalkyl, a cycloalkene, a cycloalkyne, an aryl, a heteroaryl or a heterocycloalkyl. Examples of bridged bicyclic ring systems include, but are not limited to, bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane bicyclo[3.3.3]undecane, azabicyclo[3.3.1]nonane, 3-azabicyclo[3.3.1]nonane, azabicyclo[3.2.1]octane, 3-azabicyclo[3.2.1]octane, 6-azabicyclo[3.2.1]octane and azabicyclo[2.2.2]octane, 2-azabicyclo[2.2.2]octane, and 2-oxabicyclo[2.2.2]octane.

Polycyclic ring systems have more than two rings (e.g., three rings resulting in a tricyclic ring system) and adjacent rings have at least one ring atom in common. Polycyclic ring systems include fused, bridged and spiro ring systems. A fused polycyclic ring system has at least two rings that have two adjacent ring atoms in common. A spiro polycyclic ring system has at least two rings that have only one ring atom in common. A bridged polycyclic ring system has at least two rings that have three or more adjacent ring atoms in common. Examples of polycyclic ring systems include, but are not limited to, tricyclo[3.3.1.0^{3.7}]nonane (noradamantane), tricyclo[3.3.1.1^{3.7}]decane (adamantane) and 2,3-dihydro-1H-phenalene.

“Cycloalkene” means an aliphatic cyclic hydrocarbon ring having one or more double bonds in the ring. “Cycloalkene” includes 3- to 12-membered unsaturated aliphatic cyclic hydrocarbon rings. Thus, “(C₃-C₇)cycloalkene” means a hydrocarbon radical of a 3- to 7-membered unsaturated aliphatic cyclic hydrocarbon ring. A (C₃-C₇) cycloalkene includes, but is not limited to cyclopropenyl, cyclobutenyl, cyclopentenyl, cyclohexenyl and cycloheptenyl.

A cycloalkene moiety can be monocyclic, fused bicyclic, bridged bicyclic, spiro bicyclic, or polycyclic. For example, monocyclic (C₃-C₈)cycloalkene means a radical having from 3 to 8 carbon atoms arranged in a monocyclic ring. Monocyclic (C₃-C₈)cycloalkene includes, but is not limited to, cyclopropenyl, cyclobutenyl, cyclopentenyl, cyclohexenyl and cycloheptenyl.

“Cycloalkyne” means an aliphatic cyclic hydrocarbon ring having one or more triple bonds in the ring. “Cycloalkyne” includes 3- to 12-membered unsaturated aliphatic cyclic hydrocarbon rings. Thus, “(C₃-C₇)cycloalkyne” means a hydrocarbon radical of a 3- to 7-membered unsaturated aliphatic cyclic hydrocarbon ring. A (C₃-C₇) cycloalkyne includes, but is not limited to cyclopropynyl, cyclobutynyl, cyclopentynyl, cyclohexynyl and cycloheptynyl.

A cycloalkyne moiety can be monocyclic, fused bicyclic, bridged bicyclic, spiro bicyclic, or polycyclic. For example, monocyclic (C₃-C₈)cycloalkyne means a radical having from 3 to 8 carbon atoms arranged in a monocyclic ring. Monocyclic (C₃-C₈)cycloalkyne includes, but is not limited to, cyclopropynyl, cyclobutynyl, cyclopentynyl, cyclohexynyl, and cycloheptynyl.

“Hetero” refers to the replacement of at least one carbon atom member in a ring system with at least one heteroatom selected from N, S, and O. “Hetero” also refers to the replacement of at least one carbon atom member in an acyclic system. A hetero ring system or a hetero acyclic system may have 1, 2, 3, 4 or 5 carbon atoms members replaced by a heteroatom.

“Heterocycloalkyl” means a cyclic 4- to 12-membered saturated aliphatic ring containing 1, 2, 3, 4 or 5 heteroatoms independently selected from N, O or S. When one heteroatom is S, it can be optionally mono- or di-oxygenated (i.e. —S(O)— or —S(O)₂—). When one heteroatom is N, it can be optionally substituted with alkyl, cycloalkyl, alkylene-cycloalkyl, heterocycloalkyl, alkylene-heterocycloalkyl, aryl, alkylene-aryl, heteroaryl, alkylene-heteroaryl, each of which can be optionally substituted with one or more halogen, ═O, hydroxy, alkoxy, haloalkyl, alkyl, etc.

A heterocycloalkyl moiety can be monocyclic, fused bicyclic, bridged bicyclic, spiro bicyclic, or polycyclic. For example, monocyclic (C₃-C₈) heterocycloalkyl means a 3- to 8 membered saturated aliphatic ring containing 1, 2, 3, 4, or 5 heteroatoms independently selected from N, O or S arranged in a monocyclic ring. Examples of monocyclic heterocycloalkyls include, but are not limited to, azetidine, pyrrolidine, piperidine, piperazine, azepane, hexahydropyrimidine, tetrahydrofuran, tetrahydropyran, morpholine, thiomorpholine, thiomorpholine 1,1-dioxide, tetrahydro-2H-1,2-thiazine, tetrahydro-2H-1,2-thiazine 1,1-dioxide, isothiazolidine, isothiazolidine 1,1-dioxide.

“Heteroaryl” or “heteroaromatic ring” means a 5- to 12-membered monovalent heteroaromatic monocyclic or bicyclic ring radical. A heteroaryl contains 1, 2, 3, 4, or 5 heteroatoms independently selected from N, O, and S. Heteroaryls include, but are not limited to furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, and thiazole. Bicyclic heteroaryl rings include, but are not limited to, bicyclo[4.4.0] and bicyclo[4.3.0] fused ring systems such as indolizine, indole, isoindole, indazole, benzimidazole, benzothiazole, purine, quinoline, isoquinoline, cinnoline, phthalazine, quinazoline, quinoxaline, benzofuran, 1,8-naphthyridine, and pteridine.

In a particular embodiment, each cycloalkyl, cycloalkene, cycloalkyne, cycloheterocycloalkyl, aryl and heteroaryl is optionally and independently substituted with 1 to 4. Exemplary substituents include, but are not limited to, halo, —(C₁-C₄)alkyl, —OH, ═O, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, and —C(O)—(C₁-C₄)alkyl.

“Halogen,” as used herein, refers to fluorine, chlorine, bromine, or iodine.

“Alkoxy” refers to the group —O—R where R is “alkyl”, “cycloalkyl”, “alkenyl”, or “alkynyl”. “(C₁-C₆)alkoxy” includes methoxy, ethoxy, ethenoxy, propoxy, butoxy, pentoxy, and the like.

Haloalkyl and halocycloalkyl include mono, poly, and perhalo-substituted alkyl or cycloalkyl groups where each halogen is independently selected from fluorine, chlorine, and bromine.

“Halogen” and “halo” are interchangeably used herein and each refers to fluorine, chlorine, bromine, or iodine.

“Fluoro” means —F.

As used herein, fluoro-substituted (C₁-C₄)alkyl means a (C₁-C₄)alkyl substituted with one or more —F groups. Examples of fluoro-substituted-(C₁-C₄)alkyl include, but are not limited to, —CF₃, —CH₂CF₃, —CH₂CF₂H, —CH₂CH₂F and —CH₂CH₂CF₃.

“Naturally occurring amino acid side chain moiety” refers to any amino acid side chain moiety present in a natural amino acid.

The term “pharmaceutically acceptable salt” also refers to a salt prepared from a compound disclosed herein, or any other compound delineated herein (e.g., a compound of Formulas having a basic functional group, such as an amino functional group, and a pharmaceutically acceptable inorganic or organic acid. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

The term “pharmaceutically acceptable salt” also refers to a salt prepared from a compound disclosed herein (e.g., a compound of Formulas I-III) or any other compound delineated herein, having an acidic functional group, such as a carboxylic acid functional group, and a pharmaceutically acceptable inorganic or organic base.

Salts of the compounds used in the methods of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

The invention also includes various isomers of the compounds disclosed herein and mixtures thereof. Certain compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomers” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S, either a pure enantiomer or a mixture of both configurations is present.

“Racemate” or “racemic mixture” means a compound of equimolar quantities of two enantiomers, wherein such mixtures exhibit no optical activity (i.e., they do not rotate the plane of polarized light).

The compounds of the present invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer that is present divided by the combined weight of the enantiomer that is present and the weight of its optical isomer.

As used herein, the term “tautomers” refers to isomers of organic molecules that readily interconvert by tautomerization, in which a hydrogen atom or proton migrates in the reaction, accompanied in some occasions by a switch of a single bond and an adjacent double bond.

Compounds useful for practicing the methods described herein are described in the following paragraphs, for example with references to structural formulas reproduced below. Values and alternative values for the variables in structural formulas reproduced below or an enantiomer, a diastereomer, a tautomer, or a pharmaceutically acceptable salt thereof and for each of the embodiments described herein are provided in the following paragraphs. It is understood that the invention encompasses all combinations of the substituent variables (i.e., R₁, R₂, R₃, etc.) defined herein.

Example BET Inhibitors—Structural Formulas (I) through (VIII)

In an example embodiment, bromodomain inhibitors for use in the methods of the invention, as well as methods of preparing same, are described, for example, in the U.S. Pat. No. 8,981,083, and in the International Application PCT/US2015/018118, filed on Feb. 27, 2015, published as WO 2015/131113. The teachings of this publication are incorporated herein by reference in its entirety.

Example compounds suitable for use with the methods of the present invention include those represented by structural formulas (I) through (VIII) or a pharmaceutically acceptable salt thereof. Values and alternative values for the variables in Formulas (I)-(VIII) or an enantiomer, a diastereomer, or a pharmaceutically acceptable salt thereof, and for each of the embodiments described herein are provided in the following paragraphs. It is understood that the invention encompasses all combinations of the substituent variables (i.e., R₁, R₂, R₃, etc.) defined herein.

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally substituted with 1 to 4 substituents independently selected from —F, —Cl, —Br, —OH, ═O, —S(O)—, —S(O)₂—, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₃ is selected from the group consisting of: H and —(C₁-C₄)alkyl. Further, R₃ is selected from the group consisting of: H, methyl, ethyl, propyl, butyl, sec-butyl and tert-butyl. Specifically, R₃ is H or methyl.

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

Alternatively, R_{B} is H, —(C₁-C₄)alkyl, or —(C₁-C₄)alkylene-O—(C₁-C₄)alkyl, wherein each is optionally substituted with 1 to 4 substituents independently selected from the group consisting of —F, —Cl, —Br, —OH, and —NR₅R₆.

Further, R_{B} is H, methyl, ethyl, propyl, butyl, sec-butyl, tert-butyl, —COOH, —COOMe, —COOEt, —COOCH₂OC(O)CH₃, trifluoromethyl, —CF₂—CF₃, methoxymethyl, methoxyethyl, methoxypropyl, ethoxymethyl, ethoxyethyl, methoxytrifluoromethyl, —CH₂—O—CF₂—CF₃, hydroxymethyl, hydroxyethyl, —CH₂—NH₂, —(CH₂)₂—NH₂, —CH₂—NHCH₃, or —(CH₂)₂—NHCH₃. In another alternative, R_{B} is H, methyl, ethyl, trifluoromethyl, methoxymethyl, ethoxymethyl, hydroxymethyl, hydroxyethyl, —CH₂—NH₂, or —(CH₂)₂—NH₂.

Specifically, R_{B} is H, methyl, ethyl, trifluoromethyl, methoxymethyl, ethoxymethyl, hydroxymethyl, or —CH₂—NH₂. Alternatively, R_{B} is H.

Ring A is —(C₆-C₁₀)aryl or —(C₅-C₁₀)heteroaryl. Alternatively, ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

Alternatively, ring A is 5- or 6-membered aryl or heteroaryl. Ring A is thiofuranyl, phenyl, pyridyl, furanyl, indolyl, pyrimidinyl, pyridizinyl, pyrazinyl, imidazolyl, oxazolyl, thienyl, thiazolyl, triazolyl, isoxazolyl, pyrrolyl, or pyrazolyl. Further, ring A is phenyl or thienyl. Specifically, ring A is thienyl.

Each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally substituted with 1 to 4 substituents independently selected from —F, —Cl, —Br, —OH, ═O, —S(O)—, —S(O)₂—, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl); or any two R_{A} together with the atoms to which each is bound form a fused aryl or heteroaryl group.

Alternatively, each R_{A} is independently H or —(C₁-C₄)alkyl. Each R_{A} is independently H, methyl, ethyl, propyl, butyl, sec-butyl, or tert-butyl. Specifically, each R_{A} is independently H or methyl.

Alternatively, any two R_{A} together with the atoms to which each is bound form a fused aryl or heteroaryl group. Further, any two R_{A} together with the atoms to which each is bound form a fused aryl.

R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ₒ—(C₁-C₄)alkyl, —NR₇R₈ and CN.

Alternatively, R is —(C₆-C₁₀)aryl, or —(C₅-C₁₀)heteroaryl, wherein each is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ₒ—(C₁-C₄)alkyl, —NR₇R₈ and CN.

R is phenyl or pyridinyl, wherein each is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ₒ—(C₁-C₄)alkyl, —NR₇R₈ and CN.

Further, R is phenyl or pyridinyl wherein each is optionally substituted with 1 to 4 substituents independently selected from the group consisting of: —F, —Cl, —Br, —OH, —methyl, ethyl, propyl, butyl, sec-butyl, tert-butyl, —COOH, —COOMe, —COOEt, —COOCH₂OC(O)CH₃, trifluoromethyl, —CF₂—CF₃, methoxymethyl, methoxyethyl, methoxypropyl, ethoxymethyl, ethoxyethyl, methoxytrifluoromethyl, —CH₂—O—CF₂—CF₃, hydroxymethyl, hydroxyethyl, —CH₂—NH₂, —(CH₂)₂—NH₂, —CH₂—NHCH₃, —(CH₂)₂—NHCH₃ and CN. Alternatively, R is phenyl or pyridinyl wherein each is optionally substituted with 1 to 4 substituents independently selected from the group consisting of: —F, —Cl, —Br, -and OH.

R is phenyl optionally substituted with 1 to 4 substituents independently selected from the group consisting of: —F, —Cl, —Br, -and OH. Alternatively, R is phenyl optionally substituted with 1 to 3 substituents independently selected from the group consisting of: —F, —Cl, —Br, -and OH. Further, R is phenyl optionally substituted with a substituent independently selected from the group consisting of: —F, —Cl, —Br, -and OH. Specifically, R is p-Cl-phenyl, o-Cl-phenyl, m-Cl-phenyl, p-F-phenyl, o-F-phenyl, m-F-phenyl or pyridinyl.

R₁ is —(CH₂)ₙ-L, in which n is 0-3 and L is H, —C(O)O—R₉, —CO—N(R₉R₁₀), —NR₉R₁₀, —N(R₁₀)C(O)OR₉, or —N(R₁₀)C(O)R₉.

Alternatively, R₁ is —(CH₂)ₙ-L, in which n is 0-3, and L is —C(O)O—R₉. R₁ is —(CH₂)ₙ-L, in which n is 1-3, and L is —C(O)O—R₉. Further, R₁ is —(CH₂)ₙ-L, in which n is 1-2, and L is —C(O)O—R₉. Alternatively, R₁ is —(CH₂)ₙ-L, in which n is 1, and L is —C(O)O-R₉.

Further, R₁ is —(CH₂)ₙ-L, in which n is 0-3, and L is —CO—N(R₉R₁₀). R₁ is —(CH₂)ₙ-L, in which n is 1-3, and L is —CO—N(R₉R₁₀). R₁ is —(CH₂)ₙ-L, in which n is 1-2, and L is —CO—N(R₉R₁₀). Alternatively, R₁ is —(CH₂)ₙ-L, in which n is 1, and L is —CO—N(R₉R₁₀).

In another alternative, R₁ is —(CH₂)ₙ-L, in which n is 0-3, and L is —NR₉R₁₀. R₁ is —(CH₂)ₙ-L, in which n is 1-3, and L is —NR₉R₁₀. Further, R₁ is —(CH₂)ₙ-L, in which n is 1-2, and L is —NR₉R₁₀. Alternatively, R₁ is —(CH₂)ₙ-L, in which n is 1, and L is —NR₉R₁₀.

R₁ is —(CH₂)ₙ-L, in which n is 0-3, and L is —N(R₁₀)C(O)OR₉. Alternatively, R₁ is —(CH₂)ₙ-L, in which n is 1-3, and L is —N(R₁₀)C(O)OR₉. Further, R₁ is —(CH₂)ₙ-L, in which n is 1-2, and L is —N(R₁₀)C(O)OR₉. Alternatively, R₁ is —(CH₂)ₙ-L, in which n is 1, and L is —N(R₁₀)C(O)OR₉.

Further, R₁ is —(CH₂)ₙ-L, in which n is 0-3, and L is —N(R₁₀)C(O)R₉. Alternatively, R₁ is —(CH₂)ₙ-L, in which n is 1-3, and L is —N(R₁₀)C(O)R₉. Further, R₁ is —(CH₂)ₙ-L, in which n is 1-2, and L is —N(R₁₀)C(O)R₉. Alternatively, R₁ is —(CH₂)ₙ-L, in which n is 1, and L is —N(R₁₀)C(O)R₉.

Alternatively, R₁ is —(CH₂)ₙ-L, in which n is 0-3 and L is H. R₁ is methyl, ethyl, propyl, iso-propyl. Specifically, R₁ is methyl.

R₂ is H, D, halogen, or —(C₁-C₄)alkyl. Alternatively, R₂ is H or —(C₁-C₄)alkyl. Further, R₂ is H, methyl, ethyl, propyl, iso-propyl, butyl, sec-butyl or tert-butyl. Specifically, R₂ is H or methyl.

R₄ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₄ is selected from the group consisting of: H and —(C₁-C₄)alkyl, wherein each —(C₁-C₄)alkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

R₄ is selected from the group consisting of: H and —(C₁-C₄)alkyl, wherein each —(C₁-C₄)alkyl is optionally substituted with 1 to 4 substituents independently selected from the group consisting of: —F, —Cl, —Br, and —OH. In another alternative, R₄ is selected from the group consisting of: H, methyl, ethyl, propyl, iso-propyl, butyl, sec-butyl, tert-butyl, trifluoromethyl, —CF₂—CF₃, hydroxymethyl, and hydroxyethyl. Alternatively, R₄ is selected from the group consisting of: H, methyl, ethyl, tert-butyl, and trifluoromethyl.

R₅ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₅ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₅ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₅ is selected from the group consisting of: H, methyl, ethyl, propyl, iso-propyl, butyl, tert-butyl, methoxy, hydroxyl, cyclobutyl, cyclopentyl, and cyclohexyl.

R₆ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₆ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₆ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₆ is selected from the group consisting of: H, methyl, ethyl, propyl, iso-propyl, butyl, tert-butyl, methoxy, hydroxyl, cyclobutyl, cyclopentyl, and cyclohexyl.

R₇ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₇ is selected from the group consisting of: H, —(C₁-C₄)alkyl, and —(C₃-C₈)cycloalkyl wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₇ is selected from the group consisting of: H, —(C₁-C₄)alkyl, and —(C₃-C₈)cycloalkyl, wherein each —(C₁-C₄)alkyl, and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₇ is selected from the group consisting of: H, methyl, ethyl, propyl, iso-propyl, butyl, tert-butyl, methoxy, hydroxyl, cyclobutyl, cyclopentyl, and cyclohexyl.

R₈ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₈ is selected from the group consisting of: H, —(C₁-C₄)alkyl, and —(C₃-C₈)cycloalkyl wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₈ is selected from the group consisting of: H, —(C₁-C₄)alkyl, and —(C₃-C₈)cycloalkyl, wherein each —(C₁-C₄)alkyl, and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₈ is selected from the group consisting of: H, methyl, ethyl, propyl, iso-propyl, butyl, tert-butyl, methoxy, hydroxyl, cyclobutyl, cyclopentyl, and cyclohexyl.

R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl, —(C₀-C₆)alkylene-, -cycloalkyl, -heterocycloalkyl, -aryl, and -heteroaryl is optionally substituted with 1 to 4 substituents independently selected from the group consisting of: —F, —Cl, —Br, —OH, ═O, —B(OH)₂, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ₚ—(C₁-C₄)alkyl, —NR₁₃R₁₄, and CN.

Alternatively, R₉ is selected from the group consisting of: H, —(C₁-C₆)alkyl, —(C₀-C₆)alkylene-heterocycloalkyl, —(C₀-C₆)alkylene-aryl, and —(C₀-C₆)alkylene-heteroaryl, wherein each —(C₁-C₆)alkyl, —(C₀-C₆)alkylene-, -heterocycloalkyl, -aryl, and -heteroaryl is optionally substituted with 1 to 4 substituents independently selected from the group consisting of: —F, —Cl, —Br, —OH, ═O, —B(OH)₂, —(C₁-C₄)alkyl, halo-substituted-(C₁-C₄)alkyl, —C(O)—(C₁-C₄)alkyl, and —C(O)-(fluoro-substituted-(C₁-C₄)alkyl). Further, R₉ is selected from the group consisting of: H, —(C₁-C₄)alkyl, —(C₁-C₃)alkylene-heterocycloalkyl, —(C₁-C₃)alkylene-aryl, and —(C₁-C₃)alkylene-heteroaryl, wherein each —(C₁-C₄)alkyl, —(C₁-C₃)alkylene-, -heterocycloalkyl, -aryl, and -heteroaryl is optionally substituted with 1 to 3 substituents independently selected from the group consisting of —F, —Cl, —Br, —OH, ═O, —B(OH)₂, —(C₁-C₄)alkyl, halo-substituted-(C₁-C₄)alkyl, —C(O)—(C₁-C₄)alkyl,and —C(O)-(fluoro-substituted-(C₁-C₄)alkyl).

Further, R₉ is selected from the group consisting of: H, methyl, ethyl, propyl, propyl, butyl, sec-butyl, t-butyl, and trifluoromethyl. Alternatively, R₉ is selected from the group consisting of —(C₁-C₃)alkylene-morpholine, —(C₁-C₃)alkylene-piperazine, —(C₁-C₃)alkylene-phenyl, —(C₁-C₃)alkylene-pyridyl, —(C₁-C₃)alkylene-imidazolyl, —(C₁-C₃)alkylene-azetidine, —(C₁-C₃)alkylene-furanyl, —(C₁-C₃)alkylene-pyrazinyl, —(C₁-C₃)alkylene-oxazolyl, —(C₁-C₃)alkylene-thienyl, —(C₁-C₃)alkylene-thiazolyl, —(C₁-C₃)alkylene-triazolyl, and —(C₁-C₃)alkylene-isoxazolyl, wherein each —(C₁-C₃)alkylene-, -morpholine, -piperazine, -phenyl, -pyridyl, and -imidazolyl is optionally substituted with 1 to 4 substituents independently selected from the group consisting of: —F, —Cl, OH, ═O, —B(OH)₂, —(C₁-C₄)alkyl, —C(O)—(C₁-C₄)alkyl, and halo-substituted-(C₁-C₄)alkyl.

In another alternative, R₉ is selected from the group consisting of —(C₁-C₃)alkylene-morpholine, —(C₁-C₃)alkylene-piperazine, —(C₁-C₃)alkylene-phenyl, —(C₁-C₃)alkylene-pyridyl, and —(C₁-C₃)alkylene-imidazolyl, wherein each —(C₁-C₃)alkylene-, -morpholine, -piperazine, -phenyl, -pyridyl, and -imidazolyl is optionally substituted with 1 to 4 substituents independently selected from the group consisting of: —F, —Cl, OH, ═O, —B(OH)₂, —(C₁-C₄)alkyl, —C(O)—(C₁-C₄)alkyl, and halo-substituted-(C₁-C₄)alkyl. Further, R₉ is selected from the group consisting of —(C₁-C₃)alkylene-morpholine, —(C₁-C₃)alkylene-piperazine, —(C₁-C₃)alkylene-phenyl, —(C₁-C₃)alkylene-pyridyl, and —(C₁-C₃)alkylene-imidazolyl, wherein each —(C₁-C₃)alkylene-, -morpholine, -piperazine, -phenyl, -pyridyl, and -imidazolyl is optionally substituted with 1 to 4 substituents independently selected from the group consisting of —B(OH)₂, and —(C₁-C₄)alkyl.

Alternatively, R₉ is —N═CR₁₁R₁₂.

R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl, —(C₀-C₆)alkylene-, -cycloalkyl, -heterocycloalkyl, -aryl, and -heteroaryl is optionally substituted with 1 to 4 substituents independently selected from the group consisting of: —F, —Cl, —Br, —OH, ═O, —B(OH)₂, (C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)_{q}—(C₁-C₄)alkyl, —NR₁₅R₁₆ and CN.

Alternatively, R₁₀ is selected from the group consisting of: H, —(C₁-C₆)alkyl, and —(C₁-C₆)alkylene-heterocycloalkyl, wherein each —(C₁-C₆)alkyl, —(C₁-C₆)alkylene-, and -heterocycloalkyl is optionally substituted with 1 to 4 substituents independently selected from the group consisting of: —F, —Cl, —Br, —OH, ═O, —B(OH)₂, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)_{q}—(C₁-C₄)alkyl, —NR₁₅R₁₆ and CN.

Further, R₁₀ is selected from the group consisting of: H, —(C₁-C₆)alkyl, and —(C₁-C₃)alkylene-heterocycloalkyl, wherein each —(C₁-C₆)alkyl, —(C₁-C₆)alkylene-, and -heterocycloalkyl is optionally substituted with 1 to 3 substituents independently selected from the group consisting of: —F, —Cl, —Br, —OH, ═O, —B(OH)₂, —(C₁-C₄)alkyl, halo-substituted-(C₁-C₄)alkyl, —C(O)—(C₁-C₄)alkyl,and —C(O)-(fluoro-substituted-(C₁-C₄)alkyl). Alternatively, Further, R₁₀ is selected from the group consisting of: H, methyl, ethyl, propyl, iso-propyl, butyl, sec-butyl, tert-butyl, trifluoromethyl, —(C₁-C₃)alkylene-morpholine, —(C₁-C₃)alkylene-piperazine, —(C₁-C₃)alkylene-phenyl, —(C₁-C₃)alkylene-pyridyl, and —(C₁-C₃)alkylene-imidazolyl, wherein each —(C₁-C₃)alkylene-, -morpholine, -piperazine, -phenyl, -pyridyl, and -imidazolyl is optionally substituted with 1 to 4 substituents independently selected from the group consisting of —B(OH)₂, and —(C₁-C₄)alkyl.

R₉ and R₁₀ are taken together with the nitrogen atom to which they are bound form a 4-10-membered ring. Alternatively, R₉ and R₁₀ are taken together with the nitrogen atom to which they are bound form a 4-6-membered ring. Further, R₉ and R₁₀ are taken together with the nitrogen atom to which they are bound form a 4-6-membered ring cycloalkyl or heterocycloalkyl.

R₁₁ is H, —(C₁-C₄)alkyl, or —(C₁-C₄)alkylene-O—(C₁-C₄)alkyl, wherein each —(C₁-C₄)alkyl and —(C₁-C₄)alkylene-O—(C₁-C₄)alkyl is optionally substituted with 1 to 3 substituents independently selected from the group consisting of: —F, —Cl, —Br, and —OH. Alternatively, R₁₁ is H or —(C₁-C₄)alkyl optionally substituted with 1 to 3 substituents independently selected from the group consisting of: —F, —Cl, —Br, and —OH. Further, R₁₁ is H, methyl, ethyl, propyl, butyl, or trifluoromethyl. Specifically, R₁₁ is H or methyl.

R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, ═O, —B(OH)₂, (C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ᵣ—(C₁-C₄)alkyl, —S(O)₂—Na, and CN.

Alternatively, R₁₂ is H, —(C₃-C₈)cycloalkyl, —(C₅-C₇)heterocycloalkyl, —(C₆-C₁₀)aryl, or —(C₅-C₇)heteroaryl, wherein each —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, ═O, —B(OH)₂, (C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ᵣ—(C₁-C₄)alkyl, —S(O)₂—Na, and CN. Further, R₁₂ is H, —(C₆-C₁₀)aryl, or —(C₅-C₇)heteroaryl, wherein each —(C₆-C₁₀)aryl and —(C₅-C₇)heteroaryl is optionally substituted with 1 to 4 substituents independently selected from the group consisting of: —F, —Cl, —Br, —OH, ═O, —B(OH)₂, (C1-C4)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ᵣ—(C₁-C₄)alkyl, —S(O)₂—Na, and CN.

In another alternative, R₁₂ is H, thiofuranyl, phenyl, naphthyl, biphenyl, tetrahydronaphthyl, indanyl, pyridyl, imidazolyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl, wherein each is optionally substituted with 1 to 4 substituents independently selected from the group consisting of: —F, —Cl, —Br, —OH, ═O, —B(OH)₂, (C₁-C₄)alkyl, —O—(C₁-C₄)alkyl, —S(O)ᵣ—(C₁-C₄)alkyl, —S(O)₂—Na, and CN. Alternatively, R₁₂ is H, phenyl, imidazolyl, furanyl, or indolyl, wherein each phenyl, imidazolyl, furanyl, or indolyl is optionally substituted with 1 to 4 substituents independently selected from the group consisting of: —F, —OH, methyl, —S(O)₂—Na, or —B(OH)₂,

R₁₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₁₃ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₁₃ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₁₃ is selected from the group consisting of: H, methyl, ethyl, propyl, iso-propyl, butyl, tert-butyl, methoxy, hydroxyl, cyclobutyl, cyclopentyl, and cyclohexyl.

R₁₄ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₁₄ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₁₄ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₁₄ is selected from the group consisting of: H, methyl, ethyl, propyl, iso-propyl, butyl, tert-butyl, methoxy, hydroxyl, cyclobutyl, cyclopentyl, and cyclohexyl.

R₁₅ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₁₅ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₁₅ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₁₅ is selected from the group consisting of: H, methyl, ethyl, propyl, iso-propyl, butyl, tert-butyl, methoxy, hydroxyl, cyclobutyl, cyclopentyl, and cyclohexyl.

R₁₆ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Alternatively, R₁₆ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, and —C(O)-(fluoro-substituted-(C₁-C₄)alkyl).

Further, R₁₆ is selected from the group consisting of: H, —(C₁-C₄)alkyl, and —(C₃-C₈)cycloalkyl, wherein each —(C₁-C₄)alkyl and —(C₃-C₈)cycloalkyl is optionally substituted with 1 to 4 substituents independently selected from the group consisting of: —F, —Cl, —OH, —O—(C₁-C₄)alkyl, and halo-substituted-(C₁-C₄)alkyl. In another alternative, R₁₆ is selected from the group consisting of: H, methyl, ethyl, propyl, iso-propyl, butyl, tert-butyl, methoxy, hydroxyl, cyclobutyl, cyclopentyl, and cyclohexyl.

R_{C} is selected from the group consisting of: —F, —Cl, —Br, —OH, —(C₁-C₄)alkyl, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted —(C₁-C₄)alkyl), —S(O)ₒ—(C₁-C₄)alkyl, —NR₇R₈ and CN.

Alternatively R_{C} is selected from the group consisting of: —F, —Cl, —Br, —OH, and —O—(C₁-C₄)alkyl. In another alternative, R_{C} is selected from the group consisting of F, —Cl, —Br, —OH, methoxy, and ethoxy.

m is 0, 1, 2, or 3. Alternatively, m is 1 or 2.

o is 1 or 2.

p is 1 or 2.

q is 1 or 2.

r is 1 or 2.

A first embodiment a compound is represented by Structural Formula I:

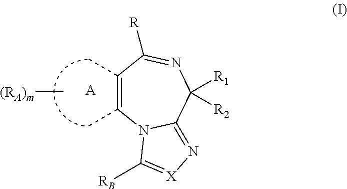

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

ring A is —(C₆-C₁₀)aryl or —(C₅-C₁₀)heteroaryl;

each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally and independently substituted with 1 to 4 substituents;

R₁ is —(CH₂)ₙ-L, in which n is 0-3 and L is H, —C(O)O—R₉, —CO—N(R₉R₁₀), —NR₉R₁₀, —N(R₁₀)C(O)OR₉, or —N(R₁₀)C(O)R₉;

R₂ is H, D, halogen, or —(C₁-C₄)alkyl;

R₄, R₅, and R₆ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₉ and R₁₀ are taken together with the nitrogen atom to which they are bound form a 4-10-membered ring;

R₁₁ is H, —(C₁-C₄)alkyl, or —(C₁-C₄)alkylene-O—(C₁-C₄)alkyl, wherein each —(C₁-C₄)alkyl and —(C₁-C₄)alkylene-O—(C₁-C₄)alkyl is optionally and independently substituted with 1 to 3 substituents selected from the group consisting of: —F, —Cl, —Br, and —OH;

R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents; and

m is 0, 1, 2, or 3.

In a first aspect of the first embodiment or the particular or specific embodiment thereof: X is N.

In a second aspect of first embodiment or the particular or specific embodiment thereof: R_{B} is H or —(C₁-C₄)alkyl.

In a third aspect of the first embodiment or the particular or specific embodiment thereof: ring A is 5- or 6-membered aryl or heteroaryl.

In a fourth aspect of the first embodiment or the particular or specific embodiment thereof: ring A is phenyl or thienyl.

In a fifth aspect of the first embodiment or the particular or specific embodiment thereof: R is —(C₆-C₁₀)aryl or —(C₅-C₁₀)heteroaryl optionally substituted with 1 to 4 substituents independently selected from the group consisting of: —F, —Cl, and —Br.

In a fifth aspect of the first embodiment or the particular or specific embodiment thereof: L is H, —COO—R₉, or —CO—N(R₉R₁₀).

In a sixth aspect of the first embodiment or the particular or specific embodiment thereof: each R₉ is independently selected from the group consisting of —(C₁-C₆)alkyl, —(C₀-C₆)alkylene-heterocycloalkyl, —(C₀-C₆)alkylene-aryl, and —(C₀-C₆)alkylene-heteroaryl and each —(C₁-C₆)alkyl, -heterocycloalkyl, -aryl, and -heteroaryl is optionally substituted with 1 to 4 substituents independently selected from the group consisting of —F, —Cl, —Br, and —(C₁-C₆)alkyl.

In a seventh aspect of the first embodiment or the particular or specific embodiment thereof: each R₁₀ is independently selected from the group consisting of: H and —(C₁-C₆)alkyl.

In an eighth aspect of the first embodiment or the particular or specific embodiment thereof: wherein R₂ is selected from the group consisting of: H and methyl.

In a ninth aspect of the first embodiment or the particular or specific embodiment thereof: R_{A} is independently H or —(C₁-C₄)alkyl, or any two R_{A} together with the atoms to which each is attached, can form a fused aryl.

In a tenth aspect of the first embodiment or the particular or specific embodiment thereof: m is 2 and and at least one R_{A} is methyl.

In an eleventh aspect of the first embodiment or the particular or specific embodiment thereof: m is 2 and each R_{A} is methyl.

In a second embodiment, a compound is represented by represented by Structural Formula II:

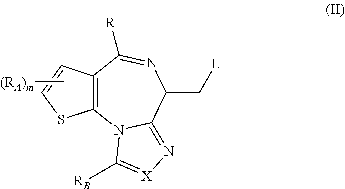

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally and independently substituted with 1 to 4 substituents;

L is H, —C(O)O—R₉, —CO—N(R₉R₁₀), —NR₉R₁₀, —N(R₁₀)C(O)OR₉, or —N(R₁₀)C(O)R₉;

R₄, R₅, and R₆ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₉ and R₁₀ are taken together with the nitrogen atom to which they are bound form a 4-10-membered ring;

R₁₁ is H, —(C₁-C₄)alkyl, or —(C₁-C₄)alkylene-O—(C₁-C₄)alkyl, wherein each-(C₁-C₄)alkyl and —(C₁-C₄)alkylene-O—(C₁-C₄)alkyl is optionally substituted with 1 to 3 substituents selected from the group consisting of: —F, —Cl, —Br, and —OH;

R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents; and

m is 0, 1, 2, or 3.

In a first aspect of the second embodiment or the particular or specific embodiment thereof: X is N.

In a second aspect of the second embodiment or the particular or specific embodiment thereof: R_{B} is selected from the group consisting of: H, —(C₁-C₄) alkyl, and —(C₁-C₄)alkylene-O—(C₁-C₄)alkyl, and each —(C₁-C₄) alkyl and —(C₁-C₄)alkylene-O—(C₁-C₄)alkyl is optionally substituted with 1 to 4 substituents independently selected from the group consisting of: —F, —Cl, —Br, and —OH.

In a third aspect of the second embodiment or the particular or specific embodiment thereof: R_{B} is methyl, ethyl, hydroxy methyl, methoxymethyl, or trifluoromethyl.

In a fourth aspect of the second embodiment or the particular or specific embodiment thereof: R is —(C₆-C₁₀)aryl, or —(C₅-C₁₀)heteroaryl optionally substituted with a substituent selected from the group consisting of: —F, —Cl, and —Br.

In a fifth aspect of the second embodiment or the particular or specific embodiment thereof: R is phenyl or pyridyl optionally substituted with a substituent selected from the group consisting of: —F, —Cl, and —Br.

In a sixth aspect of the second embodiment or the particular or specific embodiment thereof: R is p-Cl-phenyl, o-Cl-phenyl, m-Cl-phenyl, p-F-phenyl, o-F-phenyl, m-F-phenyl or pyridinyl.

In a seventh aspect of the second embodiment or the particular or specific embodiment thereof: L is —CO—N(R₉R₁₀), R₉ is —(C₀-C₆)alkylene-heterocycloalkyl, —(C₀-C₆) alkylene-aryl, or —(C₀-C₆)alkylene-heteroaryl, wherein each -heterocycloalkyl, -aryl, and -heteroaryl is optionally and independently substituted with 1 to 4 (C₁-C₄)alkyl, and R₁₀ is H or —(C₁-C₆)alkyl.

In an eighth aspect of the second embodiment or the particular or specific embodiment thereof: L is —COO—R₉ and R₉ is independently selected from the group consisting of: —(C₁-C₆)alkyl, —(C₀-C₆)alkylene -heterocycloalkyl, —(C₀-C₆)alkylene-aryl, and —(C₀-C₆)alkylene-heteroaryl, wherein each —(C₁-C₆)alkyl, -heterocycloalkyl, -aryl, and -heteroaryl is optionally substituted with 1 to 4 substituents independently selected from the group consisting of: —F, —Cl, —Br, and —(C₁-C₆)alkyl.

In a ninth aspect of the second embodiment or the particular or specific embodiment thereof: L is —COO—R₉, and R₉ is selected from the group consisting of: methyl, ethyl, propyl, i-propyl, butyl, sec-butyl, t-butyl, and trifluoromethyl.

In a tenth aspect of the second embodiment or the particular or specific embodiment thereof: each R_{A} is independently H or —(C₁-C₄)alkyl, or any two R_{A} together with the atoms to which each is attached, can form a fused aryl.

In an eleventh aspect of the second embodiment or the particular or specific embodiment thereof: m is 2, and at least one occurrence of R_{A} is methyl.

In a twelfth aspect of the second embodiment or the particular or specific embodiment thereof: m is 2 and each R_{A} is methyl.

In a third embodiment, a compound is represented by represented by Structural Formula III:

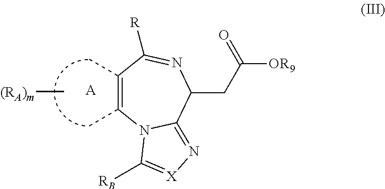

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

ring A is —(C₆-C₁₀)aryl or —(C₅-C₁₀)heteroaryl;

each R_{A} is independently H, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally and independently substituted with 1 to 4 substituents;

R₄, R₅, and R₆ are each independently selected from the group consisting of: H, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₀-C₆)alkylene-cycloalkyl, —(C₀-C₆)alkylene-heterocycloalkyl, —(C₀-C₆)alkylene-aryl, and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents; and

m is 0, 1, 2, or 3.

In a first aspect of the third embodiment or the particular or specific embodiment thereof: X is N.

In a second aspect of the third embodiment or the particular or specific embodiment thereof: R_{B} is selected from the group consisting of: H, —(C₁-C₄) alkyl, and —(C₁-C₄)alkylene-O—(C₁-C₄)alkyl, and each —(C₁-C₄) alkyl and —(C₁-C₄)alkylene-O—(C₁-C₄)alkyl is optionally substituted with 1 to 4 substituents independently selected from the group consisting of —F, —Cl, —Br, and —OH.

In a third aspect of the third embodiment or the particular or specific embodiment thereof: R_{B} is methyl, ethyl, hydroxy methyl, methoxymethyl, or trifluoromethyl.

In a fourth aspect of the third embodiment or the particular or specific embodiment thereof: ring A is 5- or 6-membered aryl or heteroaryl.

In a fifth aspect of the third embodiment or the particular or specific embodiment thereof: ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In a sixth aspect of the third embodiment or the particular or specific embodiment thereof: ring A is phenyl or thienyl.

In a seventh aspect of the third embodiment or the particular or specific embodiment thereof: R is —(C₆-C₁₀)aryl or —(C₅-C₁₀)heteroaryl optionally substituted with a substituent selected from the group consisting of: —F, —Cl, and —Br.

In an eighth aspect of the third embodiment or the particular or specific embodiment thereof: R is phenyl or pyridyl optionally substituted with 1-4 substituents independently selected from the group consisting of: —F, —Cl, and —Br.

In a ninth aspect of the third embodiment or the particular or specific embodiment thereof: R is p-Cl-phenyl, o-Cl-phenyl, m-Cl-phenyl, p-F-phenyl, o-F-phenyl, m-F-phenyl or pyridinyl.

In a tenth aspect of the third embodiment or the particular or specific embodiment thereof: each R_{A} is independently H or —(C₁-C₄)alkyl, or any two R_{A} together with the atoms to which each is attached, can form a fused aryl.

In an eleventh aspect of the third embodiment or the particular or specific embodiment thereof: m is 2, and at least one occurrence of R_{A} is methyl.

In a twelfth aspect of the third embodiment or the particular or specific embodiment thereof: m is 2 and each R_{A} is methyl.

In a thirteenth aspect of the third embodiment or the particular or specific embodiment thereof: R₉ is independently selected from the group consisting of —(C₁-C₆)alkyl, —(C₀-C₆)alkylene -heterocycloalkyl, —(C₀-C₆)alkylene-aryl, and —(C₀-C₆)alkylene-heteroaryl and each —(C₁-C₆)alkyl, -heterocycloalkyl, -aryl, and -heteroaryl is optionally substituted with 1 to 4 substituents independently selected from the group consisting of —F, —Cl, —Br, and —(C₁-C₆)alkyl.

In a fourteenth aspect of the third embodiment or the particular or specific embodiment thereof: R₉ is selected from the group consisting of: methyl, ethyl, propyl, propyl, butyl, sec-butyl, t-butyl, and trifluoromethyl.

In a fourth embodiment, a compound is represented by represented by Structural Formula IV:

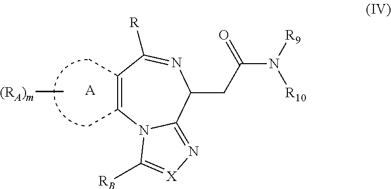

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

ring A is aryl or heteroaryl;

each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally and independently substituted with 1 to 4 substituents;

R₄, R₅, and R₆ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₁₀ is selected from the group consisting of: H, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₉ and R₁₀ are taken together with the nitrogen atom to which they are bound form a 4-10-membered ring;

R₁₁ is H, —(C₁-C₄)alkyl, or —(C₁-C₄)alkylene-O—(C₁-C₄)alkyl, wherein each —(C₁-C₄)alkyl and —(C₁-C₄)alkylene-O—(C₁-C₄)alkyl is optionally substituted with 1 to 3 substituents selected from the group consisting of: —F, —Cl, —Br, and —OH;

R₁₂ is H, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents; and

m is 0, 1, 2, or 3.

In a first aspect of the fourth embodiment or the particular or specific embodiment thereof: X is N.

In a second aspect of the fourth embodiment or the particular or specific embodiment thereof: R_{B} is selected from the group consisting of: H, —(C₁-C₄) alkyl, and —(C₁-C₄)alkylene-O—(C₁-C₄)alkyl, and each —(C₁-C₄) alkyl and —(C₁-C₄)alkylene-O—(C₁-C₄)alkyl is optionally substituted with 1 to 4 substituents independently selected from the group consisting of —F, —Cl, —Br, and —OH.

In a third aspect of the fourth embodiment or the particular or specific embodiment thereof: R_{B} is methyl, ethyl, hydroxy methyl, methoxymethyl, or trifluoromethyl.

In a fourth aspect of the fourth embodiment or the particular or specific embodiment thereof: ring A is 5- or 6-membered aryl or heteroaryl.

In a fifth aspect of the fourth embodiment or the particular or specific embodiment thereof: ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In a sixth aspect of the fourth embodiment or the particular or specific embodiment thereof: ring A is phenyl or thienyl.

In a seventh aspect of the fourth embodiment or the particular or specific embodiment thereof: R is —(C₆-C₁₀)aryl, or —(C₅-C₁₀)heteroaryl optionally substituted with 1 to 4 substituents independently selected from the group consisting of: —F, —Cl, and —Br.

In an eighth aspect of the fourth embodiment or the particular or specific embodiment thereof: R is phenyl or pyridyl optionally substituted with 1 to 4 substituents independently selected from the group consisting of: —F, —Cl, and —Br.

In a ninth aspect of the fourth embodiment or the particular or specific embodiment thereof: R is p-Cl-phenyl, o-Cl-phenyl, m-Cl-phenyl, p-F-phenyl, o-F-phenyl, m-F-phenyl or pyridinyl.

In a tenth aspect of the fourth embodiment or the particular or specific embodiment thereof: each R_{A} is independently H or —(C₁-C₄)alkyl, or any two R_{A} together with the atoms to which each is attached, can form a fused aryl.

In an eleventh aspect of the fourth embodiment or the particular or specific embodiment thereof: m is 2, and at least one occurrence of R_{A} is methyl.

In a twelfth aspect of the fourth embodiment or the particular or specific embodiment thereof: m is 2 and each R_{A} is methyl.

In a thirteenth aspect of the fourth embodiment or the particular or specific embodiment thereof: R₉ is independently selected from the group consisting of —(C₁-C₆)alkyl, —(C₀-C₆)alkylene-heterocycloalkyl, —(C₀-C₆)alkylene-aryl, and —(C₀-C₆)alkylene-heteroaryl and each —(C₁-C₆)alkyl, -heterocycloalkyl, -aryl, and -heteroaryl is optionally substituted with 1 to 4 substituents independently selected from the group consisting of —F, —Cl, —Br, and —(C₁-C₆)alkyl.

In a fourteenth aspect of the fourth embodiment or the particular or specific embodiment thereof: R₁₀ is selected from the group consisting of: H and —(C₁-C₆)alkyl optionally substituted with 1 to 4 substituents independently selected from the group consisting of —F, and —O—(C₁-C₆)alkyl.

In a fifteenth aspect of the fourth embodiment or the particular or specific embodiment thereof: R₉ is N═CR₁₁R₁₂, R₁₁ is H or —(C₁-C₄)alkyl and R₁₂ is —(C₅-C₇)heterocycloalkyl, —(C₆-C₁₀)aryl or —(C₅-C₇)heteroaryl, optionally substituted with 1 to 4 substituents independently selected from —(C₁-C₄)alkyl, —F, —Cl, —SO₂Na, or —B(OH)₂.

In a fifth embodiment, a compound is represented by represented by Structural Formula V:

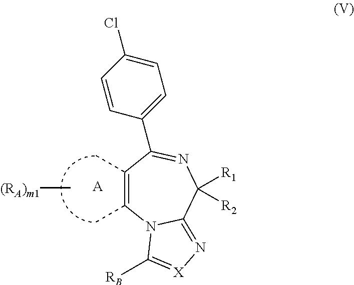

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

ring A is —(C₆-C₁₀)aryl or —(C₅-C₁₀)heteroaryl;

each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

R₁ is —(CH₂)ₙ-L, in which n is 0-3 and L is H, —C(O)O—R₉, —CO—N(R₉R₁₀), —NR₉R₁₀, —N(R₁₀)C(O)OR₉, or —N(R₁₀)C(O)R₉;

R₂ is H, D, halogen, or —(C₁-C₄)alkyl;

R₄, R₅, and R₆ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₉ and R₁₀ are taken together with the nitrogen atom to which they are bound form a 4-10-membered ring;

R₁₁ is H, —(C₁-C₄)alkyl, or —(C₁-C₄)alkylene-O—(C₁-C₄)alkyl, wherein each —(C₁-C₄)alkyl, and —(C₁-C₄)alkylene-O—(C₁-C₄)alkyl is optionally and independently substituted with 1 to 3 substituents selected from the group consisting of: —F, —Cl, —Br, and —OH;

R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents; and

m is 0, 1, 2, or 3.

In a first aspect of the fifth embodiment or the particular or specific embodiment thereof: X is N.

In a second aspect of the fifth embodiment or the particular or specific embodiment thereof: R_{B} is selected from the group consisting of: H, —(C₁-C₄) alkyl, and —(C₁-C₄)alkylene-O—(C₁-C₄)alkyl, and each —(C₁-C₄) alkyl and —(C₁-C₄)alkylene-O—(C₁-C₄)alkyl is optionally substituted with 1 to 4 substituents independently selected from the group consisting of —F, —Cl, —Br, and —OH.

In a third aspect of the fifth embodiment or the particular or specific embodiment thereof: R_{B} is methyl, ethyl, hydroxy methyl, methoxymethyl, or trifluoromethyl.

In a fourth aspect of the fifth embodiment or the particular or specific embodiment thereof: ring A is 5- or 6-membered aryl or heteroaryl.

In a fifth aspect of the fifth embodiment or the particular or specific embodiment thereof: ring A is thiofuranyl, phenyl, naphthyl, biphenyl, tetrahydronaphthyl, indanyl, pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, or 5,6,7,8-tetrahydroisoquinolinyl.

In a sixth aspect of the fifth embodiment or the particular or specific embodiment thereof: ring A is phenyl or thienyl.

In a seventh aspect of the fifth embodiment or the particular or specific embodiment thereof: R_{A} is independently H or —(C₁-C₄)alkyl, or any two R_{A} together with the atoms to which each is attached, can form a fused aryl.

In an eighth aspect of the fifth embodiment or the particular or specific embodiment thereof: m is 2, and at least one occurrence of R_{A} is methyl.

In a ninth aspect of the fifth embodiment or the particular or specific embodiment thereof: m is 2 and each R_{A} is methyl.

In a tenth aspect of the fifth embodiment or the particular or specific embodiment thereof: L is —CO—N(R₉R₁₀), R₉ is —(C₀-C₆)alkylene-heterocycloalkyl, —(C₀-C₆) alkylene-aryl, or —(C₀-C₆)alkylene-heteroaryl, optionally and independently substituted with 1 to 4 (C₁-C₄)alkyl, and R₁₀ is H or —(C₁-C₆)alkyl.

In a eleventh aspect of the fifth embodiment or the particular or specific embodiment thereof: L is —COO—R₉, and R₉ is independently selected from the group consisting of —(C₁-C₆)alkyl, —(C₀-C₆)alkylene-heterocycloalkyl, —(C₀-C₆)alkylene-aryl, and —(C₀-C₆)alkylene-heteroaryl and each —(C₁-C₆)alkyl, -heterocycloalkyl, -aryl, and -heteroaryl is optionally substituted with 1 to 4 substituents independently selected from the group consisting of —F, —Cl, —Br, and —(C₁-C₆)alkyl.

In a twelfth aspect of the fifth embodiment or the particular or specific embodiment thereof: L is —COO—R₉, and R₉ is selected from the group consisting of: methyl, ethyl, propyl, i-propyl, butyl, sec-butyl, t-butyl, and trifluoromethyl.

In a thirteenth aspect of the fifth embodiment or the particular or specific embodiment thereof: R₂ is H or —(C₁-C₄)alkyl.

In a sixth embodiment, a compound is represented by represented by Structural Formula VI:

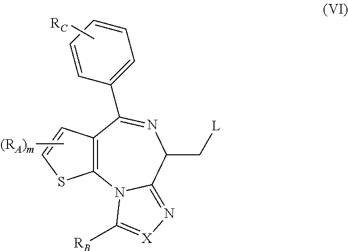

or a pharmaceutically acceptable salt thereof, wherein:

X is N or CR₃;

R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;

R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;

each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;

L is H, —C(O)O—R₉, —CO—N(R₉R₁₀), —NR₉R₁₀, —N(R₁₀)C(O)OR₉, or —N(R₁₀)C(O)R₉;

R_{C} is selected from the group consisting of: —F, —Cl, —Br, —OH, —O—(C₁-C₄)alkyl, —(C₁-C₄)alkylene-O—(C₁-C₄)alkyl, halo-substituted-(C₁-C₄)alkyl, halo-substituted-O—(C₁-C₄)alkyl, —C(O)—(C₁-C₄)alkyl, —C(O)-(fluoro-substituted-(C₁-C₄)alkyl), —S(O)ₒ—(C₁-C₄)alkyl, —NR₇R₈ and CN;

R₄, R₅, R₆, R₇ and R₈ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;

R₉ and R₁₀ are taken together with the nitrogen atom to which they are bound form a 4-10-membered ring;

R₁₁ is H, —(C₁-C₄)alkyl, or —(C₁-C₄)alkylene-O—(C₁-C₄)alkyl, wherein each —(C₁-C₄)alkyl, and —(C₁-C₄)alkylene-O—(C₁-C₄)alkyl is optionally and independently substituted with 1 to 3 substituents selected from the group consisting of: —F, —Cl, —Br, and —OH;

R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;

m is 0, 1, 2, or 3; and

o is 1 or 2.

In a first aspect of the sixth embodiment or the particular or specific embodiment thereof: X is N.

In a second aspect of the sixth embodiment or the particular or specific embodiment thereof: R_{B} is selected from the group consisting of: H, —(C₁-C₄) alkyl, and —(C₁-C₄)alkylene-O—(C₁-C₄)alkyl, and each —(C₁-C₄) alkyl and —(C₁-C₄)alkylene-O—(C₁-C₄)alkyl is optionally substituted with 1 to 4 substituents independently selected from the group consisting of —F, —Cl, —Br, and —OH.

In a third aspect of the sixth embodiment or the particular or specific embodiment thereof: R_{B} is methyl, ethyl, hydroxy methyl, methoxymethyl, or trifluoromethyl.

In a fourth aspect of the sixth embodiment or the particular or specific embodiment thereof: each R_{A} is independently H or —(C₁-C₄)alkyl, or any two R_{A} together with the atoms to which each is attached, can form a fused aryl.

In a fifth aspect of the sixth embodiment or the particular or specific embodiment thereof: m is 1 or 2, and at least one occurrence of R_{A} is methyl.

In a sixth aspect of the sixth embodiment or the particular or specific embodiment thereof: m is 2 and each R_{A} is methyl.

In a seventh aspect of the sixth embodiment or the particular or specific embodiment thereof: L is —CO—N(R₉R₁₀), R₉ is —(C₀-C₆)alkylene-heterocycloalkyl, —(C₀-C₆) alkylene-aryl, or —(C₀-C₆)alkylene-heteroaryl and each -heterocycloalkyl, -aryl, and -heteroaryl is optionally and independently substituted with 1 to 4 (C₁-C₄)alkyl, and R₁₀ is H or —(C₁-C₆)alkyl.

In an eighth aspect of the sixth embodiment or the particular or specific embodiment thereof: L is —COO—R₉, and R₉ is independently selected from the group consisting of —(C₁-C₆)alkyl, —(C₀-C₆)alkylene -heterocycloalkyl, —(C₀-C₆)alkylene-aryl, and —(C₀-C₆)alkylene-heteroaryl and each —(C₁-C₆)alkyl, -heterocycloalkyl, -aryl, and -heteroaryl is optionally substituted with 1 to 4 substituents independently selected from the group consisting of —F, —Cl, —Br, and —(C₁-C₆)alkyl.

In a ninth aspect of the sixth embodiment or the particular or specific embodiment thereof: L is —COO—R₉, and R₉ is selected from the group consisting of: methyl, ethyl, propyl, i-propyl, butyl, sec-butyl, t-butyl, and trifluoromethyl.

In a tenth aspect of the sixth embodiment or the particular or specific embodiment thereof: R_{C} is selected from the group consisting of: —F, —Cl, —Br, —OH, and —O—(C₁-C₄)alkyl.

In a seventh embodiment, a compound is represented following structural formula:

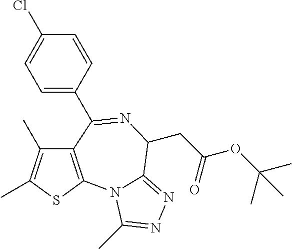 [SMILES: Cc1sc2c(c1C)C(c1ccc(Cl)cc1)=NC(CC(=O)OC(C)(C)C)c1nnc(C)n1-2]

or a pharmaceutically acceptable salt thereof.

In first aspect of the seventh embodiment or the particular or specific embodiments thereof, the compound is represented following structural formula:

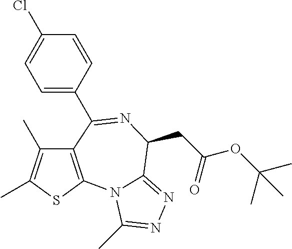 [SMILES: Cc1sc2c(c1C)C(c1ccc(Cl)cc1)=N[C@@H](CC(=O)OC(C)(C)C)c1nnc(C)n1-2]

or a pharmaceutically acceptable salt thereof.

In an eighth embodiment, a compound is represented by represented by any one of the following structural formulas:

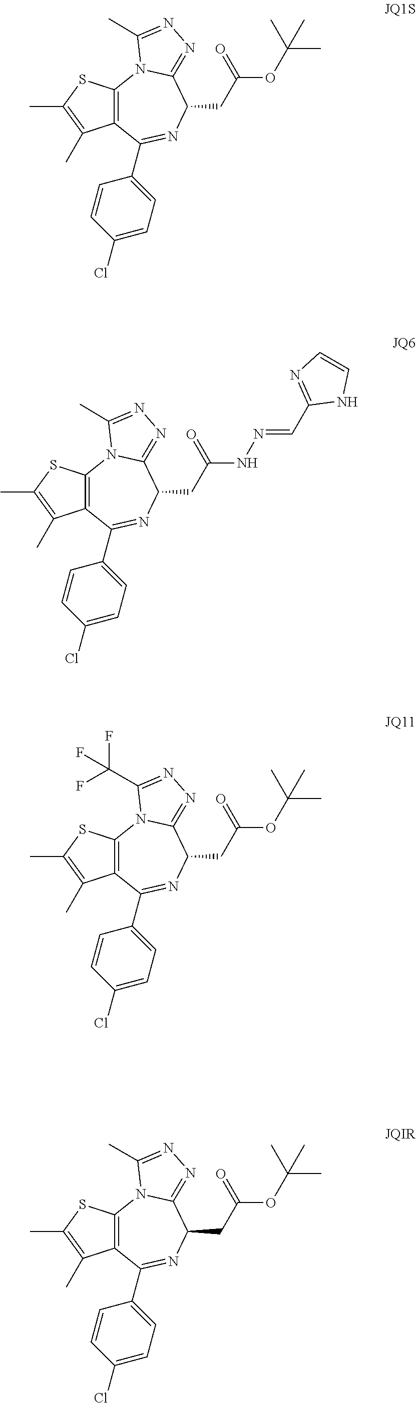

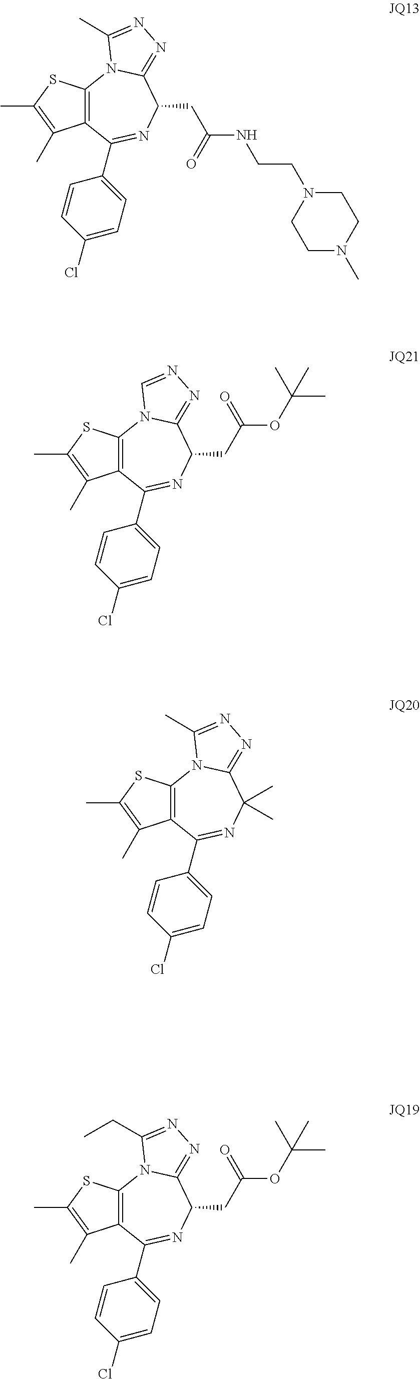

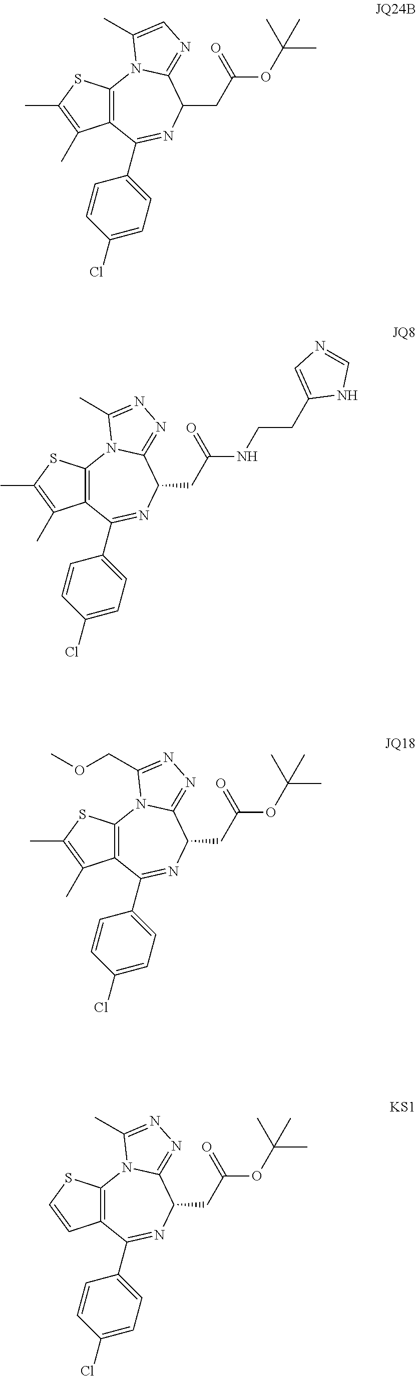

or a pharmaceutically acceptable salt thereof.

In a ninth embodiment, a compound is represented by represented by any one of the following structural formulas:

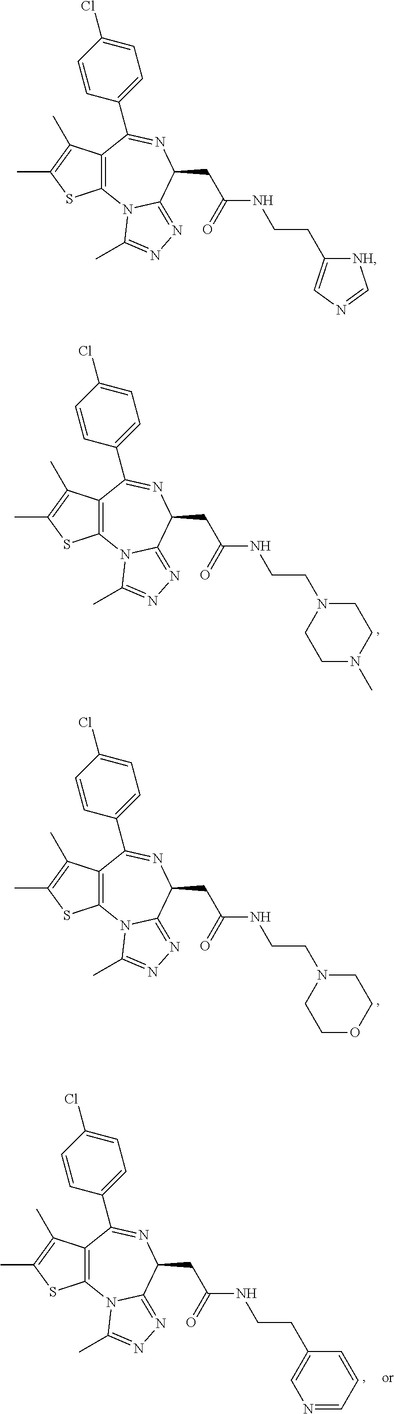

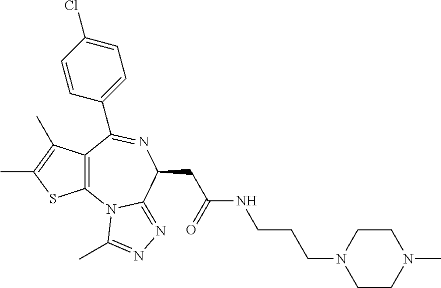

or a pharmaceutically acceptable salt thereof.

In a tenth embodiment, a compound is represented by represented by any one of the following structural formulas:

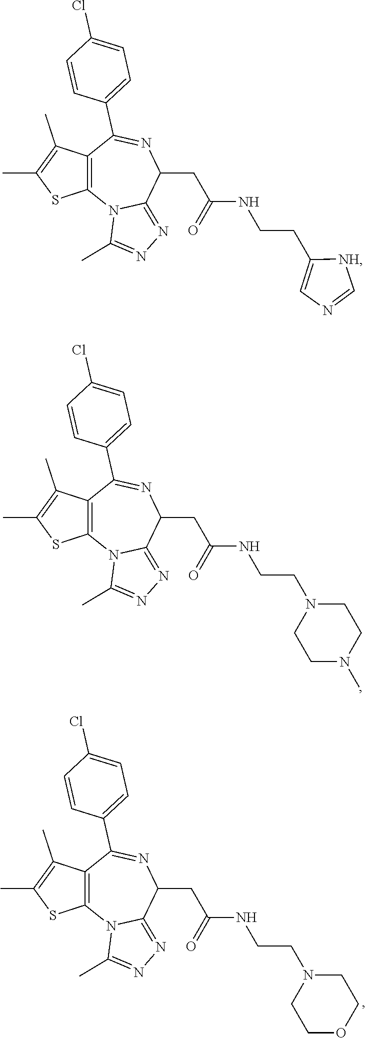

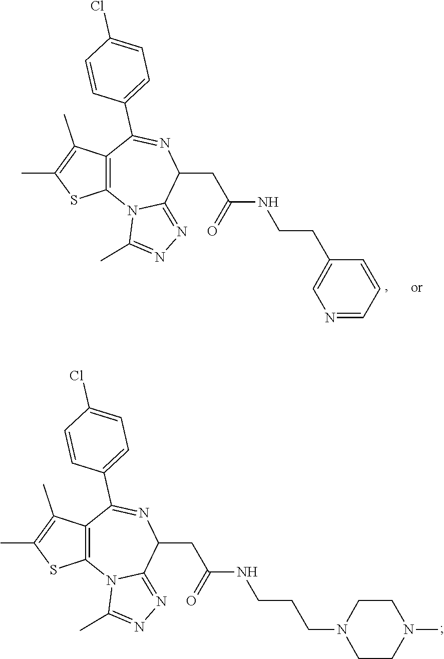

or a pharmaceutically acceptable salt thereof.

In an eleventh embodiment, a compound is represented by represented by any one of the following structural formulas:

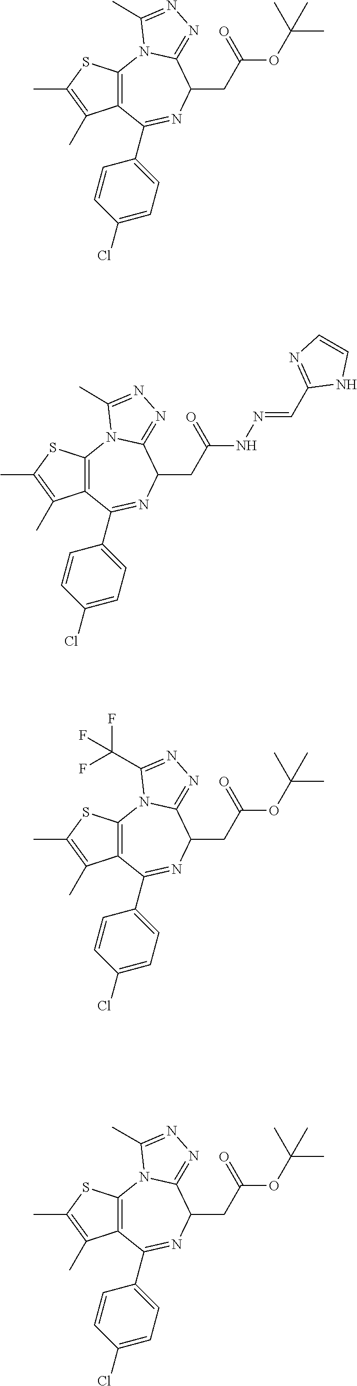

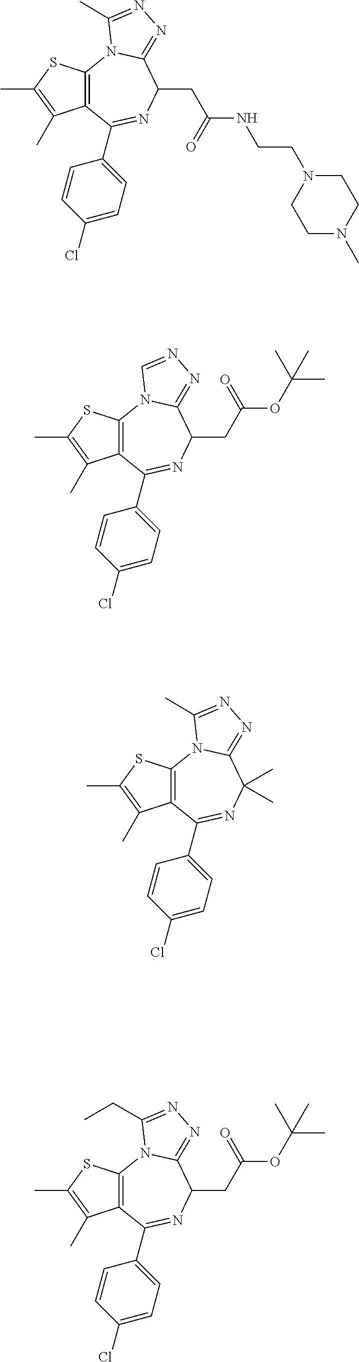

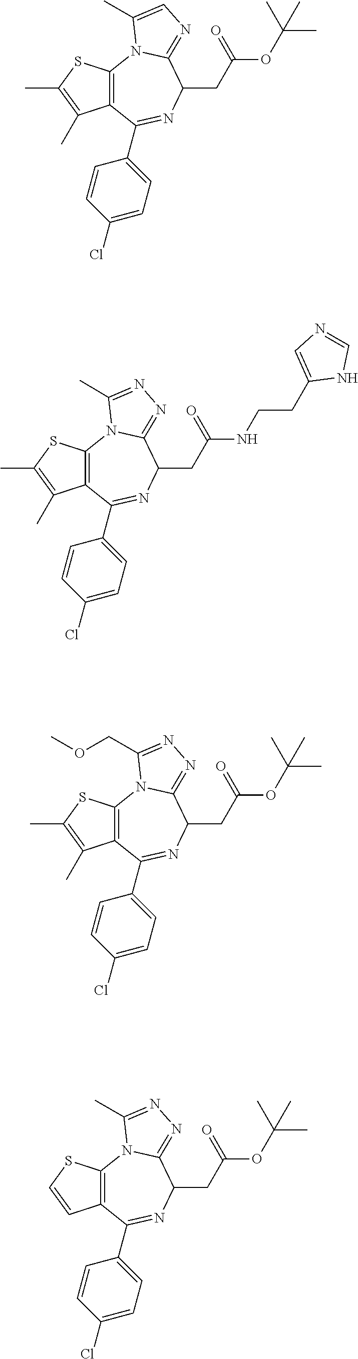

or a pharmaceutically acceptable salt thereof.

In a twelfth embodiment, a compound is represented by represented by any one of the following structural formulas:

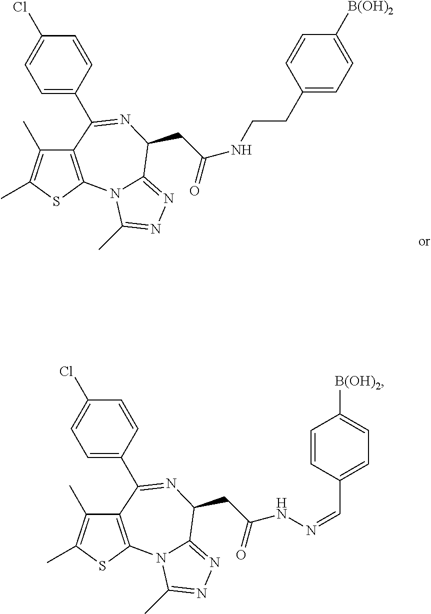 [SMILES: Cc1sc2c(c1C)C(c1ccc(Cl)cc1)=N[C@@H](CC(=O)NCCc1ccc(B(O)O)cc1)c1nnc(C)n1-2 ; Cc1sc2c(c1C)C(c1ccc(Cl)cc1)=N[C@@H](CC(=O)N/N=C\c1ccc(B(O)O)cc1)c1nnc(C)n1-2]

or a pharmaceutically acceptable salt thereof.

In a thirteenth embodiment, a compound is represented by represented by any one of the following structural formulas:

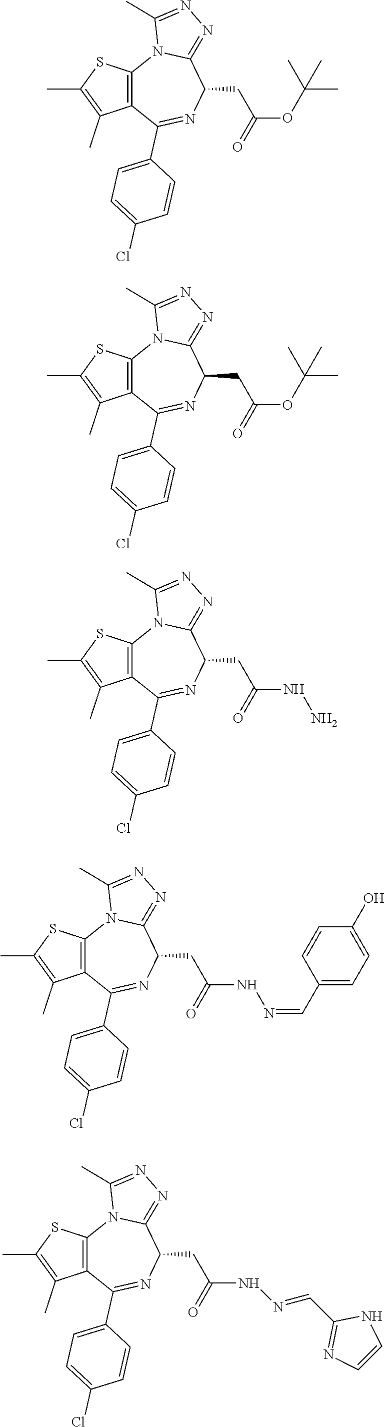

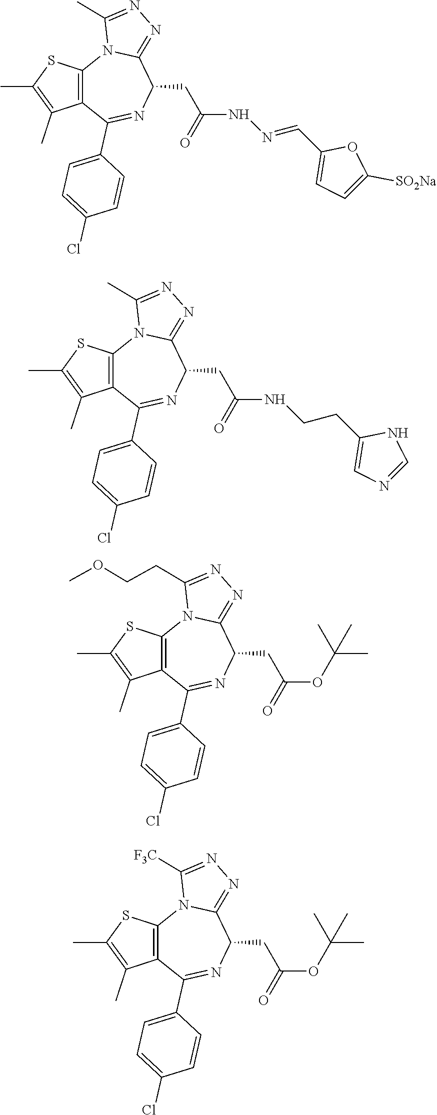

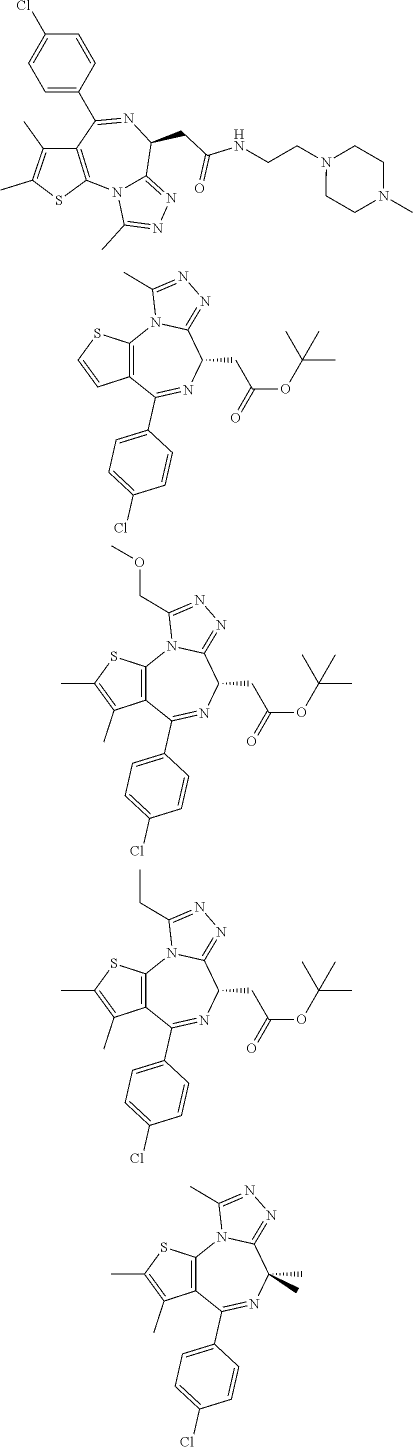

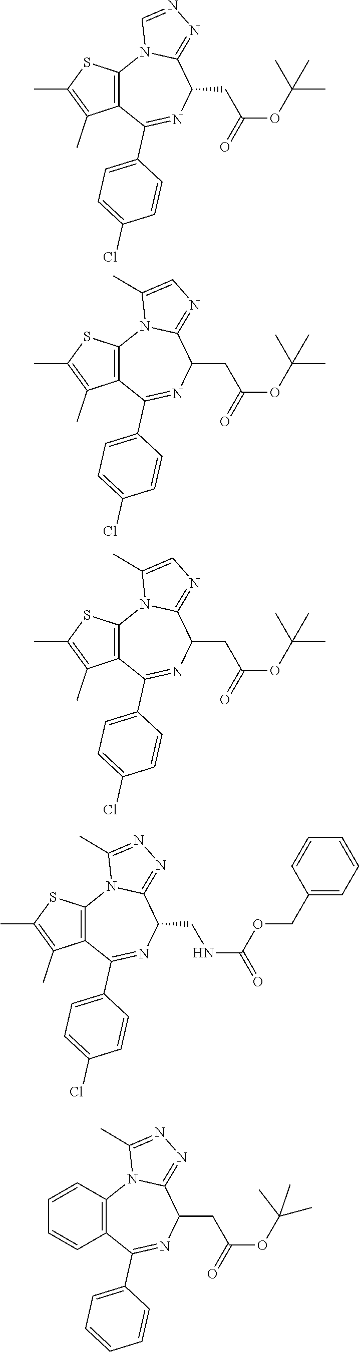

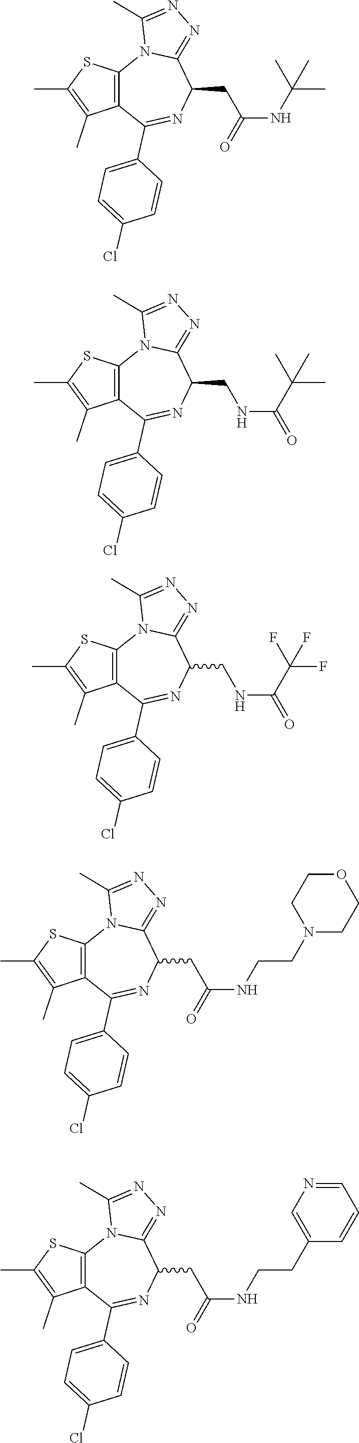

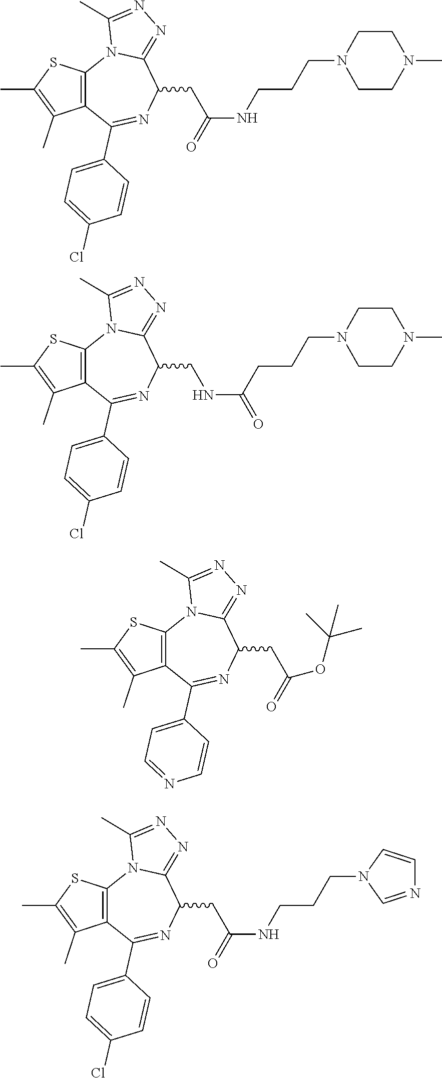

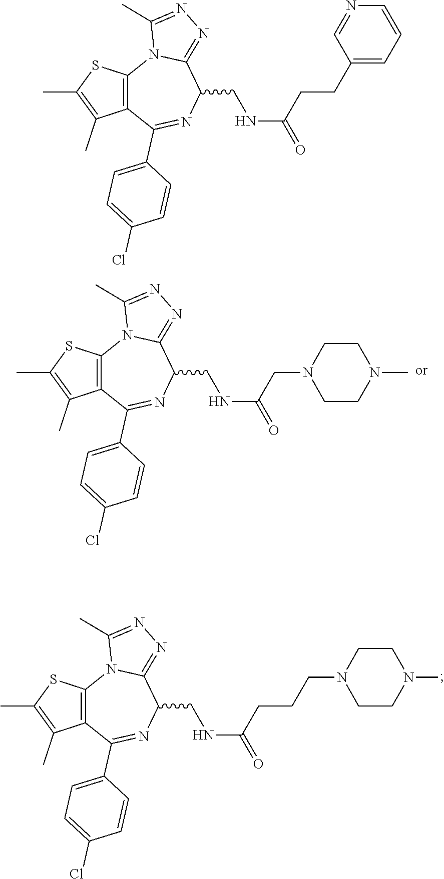

or a pharmaceutically acceptable salt thereof.

In a fourteenth embodiment, a compound is represented by represented by any one of the following structural formulas:

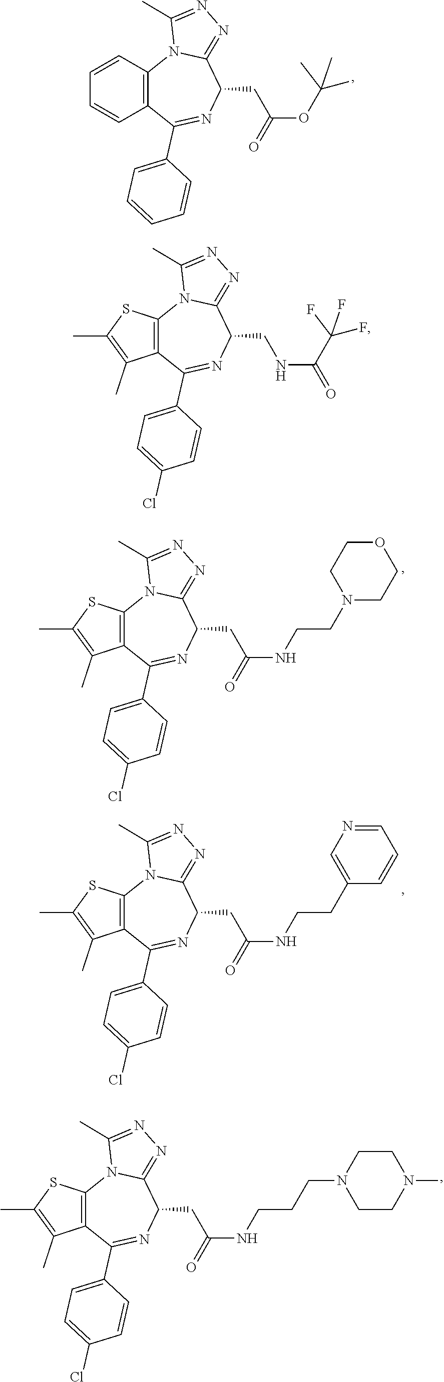

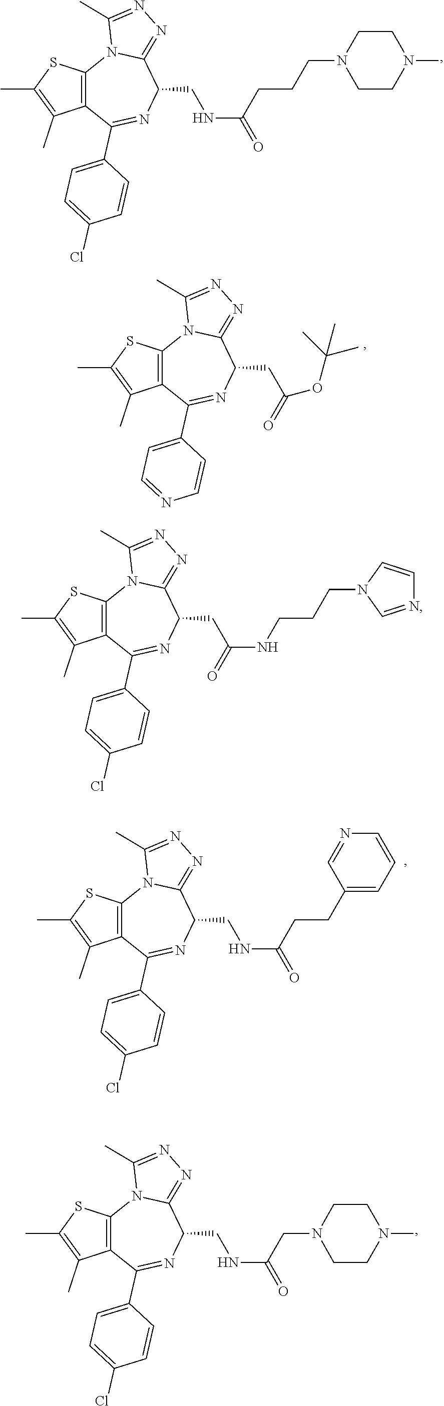

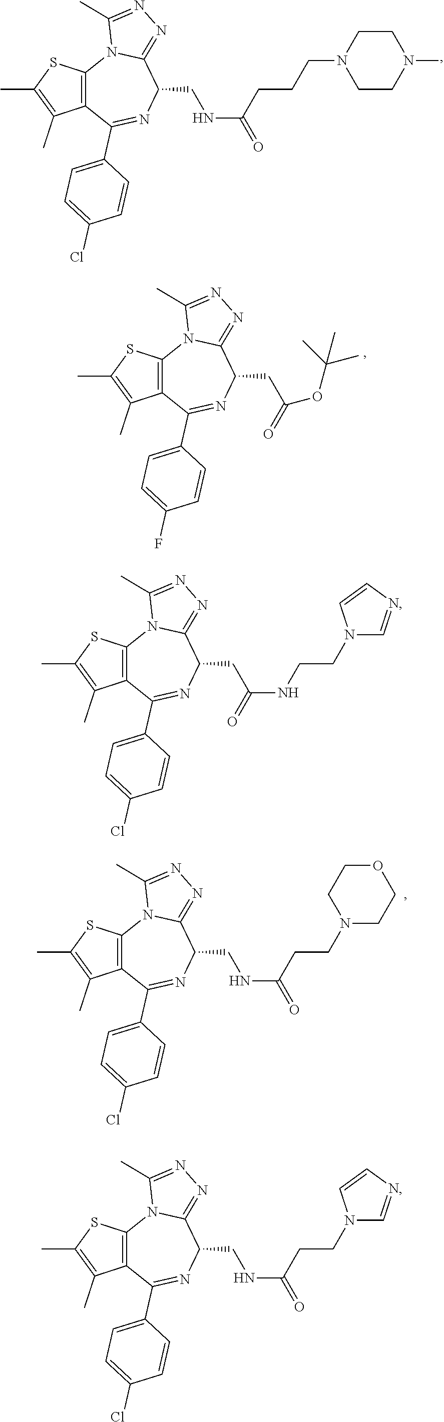

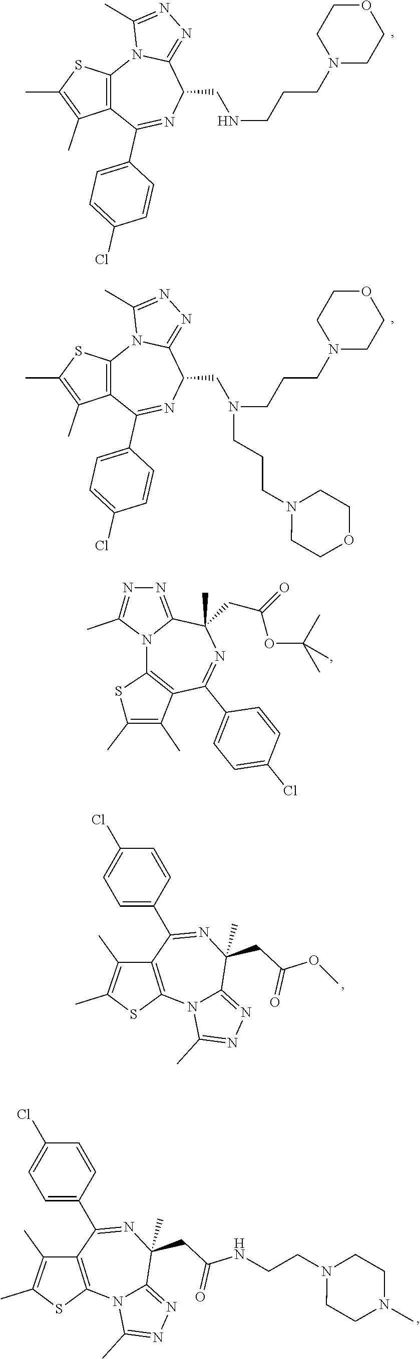

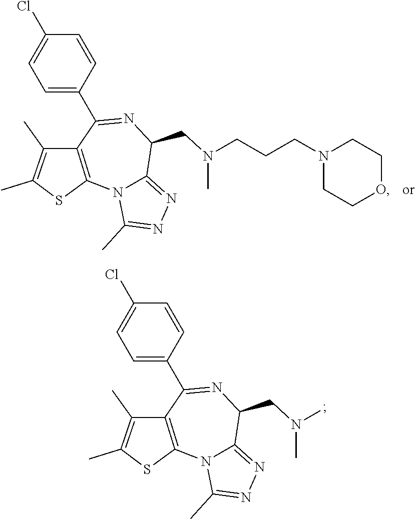

or a pharmaceutically acceptable salt thereof.

In a fifteenth embodiment, a compound is represented by the structure:

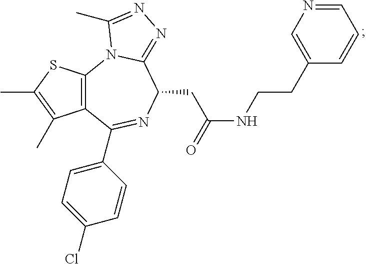 [SMILES: Cc1sc2c(c1C)C(c1ccc(Cl)cc1)=N[C@@H](CC(=O)NCCc1cccnc1)c1nnc(C)n1-2]

or a pharmaceutically acceptable salt thereof.

In a sixteenth embodiment, a compound is represented by the structure:

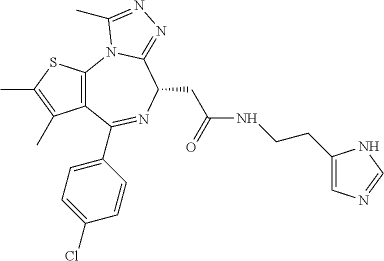 [SMILES: Cc1sc2c(c1C)C(c1ccc(Cl)cc1)=N[C@@H](CC(=O)NCCc1cnc[nH]1)c1nnc(C)n1-2]

or a pharmaceutically acceptable salt thereof.

In a seventeenth embodiment, a compound is represented by the structure:

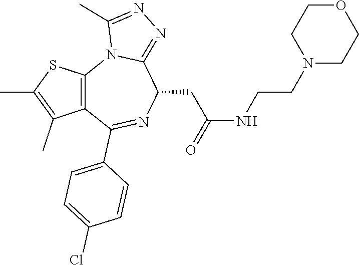 [SMILES: Cc1sc2c(c1C)C(c1ccc(Cl)cc1)=N[C@@H](CC(=O)NCCN1CCOCC1)c1nnc(C)n1-2]

or a pharmaceutically acceptable salt thereof.

In an eighteenth embodiment, a compound is represented by Structural Formula (VI), (VII), or (VIII):

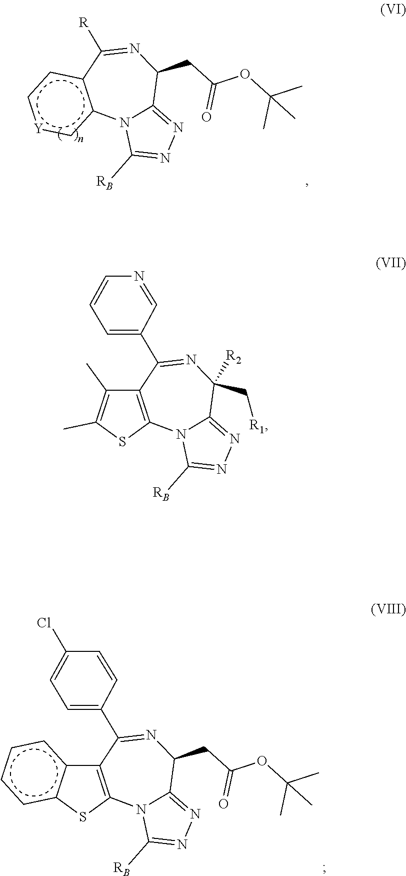

in which R, R₁, and R₂ and R_{B} have the same meaning as in Formula (I); Y is O, N, S, or CR₃, in which R₃ has the same meaning as in Formula (I); n is 0 or 1; and the dashed circle in Formula (VIII) indicates an aromatic or non-aromatic ring; or a pharmaceutically acceptable salt thereof.

In a nineteenth embodiment, a compound is represented by the structure:

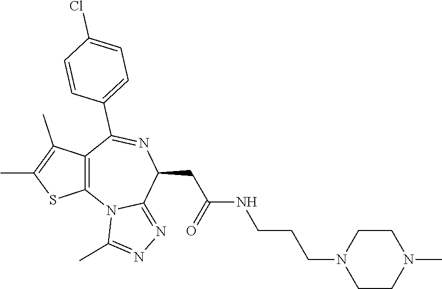

or a pharmaceutically acceptable salt thereof.

In certain embodiments, the compound for use in the methods of the invention is a compound selected from the group consisting of:

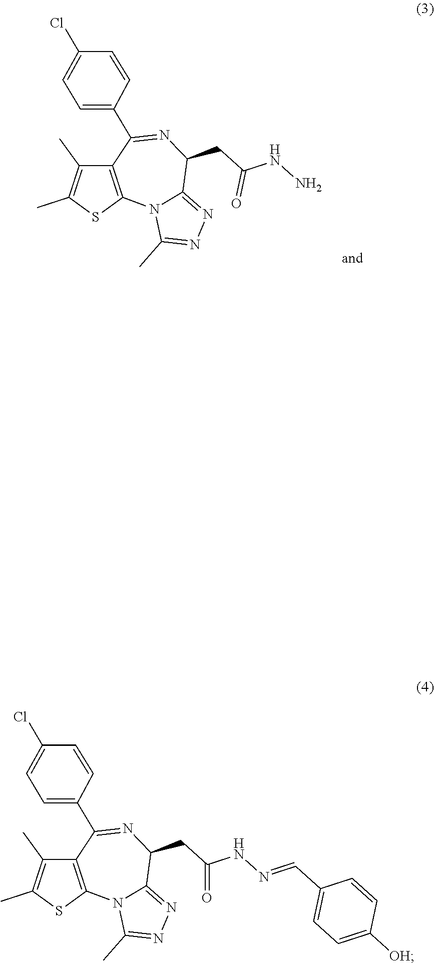 [SMILES: Cc1sc2c(c1C)C(c1ccc(Cl)cc1)=N[C@@H](CC(=O)NN)c1nnc(C)n1-2 ; Cc1sc2c(c1C)C(c1ccc(Cl)cc1)=N[C@@H](CC(=O)N/N=C/c1ccc(O)cc1)c1nnc(C)n1-2]

or a pharmaceutically acceptable salt thereof.

Example BET Inhibitors—Structural Formulas (IX) to (XI)

In another example embodiment, bromodomain inhibitors for use in the methods of the invention, as well as methods of preparing same, are described in U.S. Provisional Application No. 62/068,983, filed on Oct. 27, 2014. The teachings of this application are incorporated herein by referejce in its entirety.

Example compounds suitable for use with the methods of the present invention include those represented by structural formulas (IX), (X), and (XI), or a pharmaceutically acceptable salt thereof. Values and alternative values for the variables in Formulas (IX-XI) or an enantiomer, a diastereomer, or a pharmaceutically acceptable salt thereof, and for each of the embodiments described herein are provided in the following paragraphs. It is understood that the invention encompasses all combinations of the substituent variables (i.e., R₁, R₂, R₂₀, etc.) defined herein.

A is selected from the group consisting of a (C₁-C₆)alkyl, a (C₂-C₆)alkenyl, a (C₂-C₆)alkynyl, a (C₃-C₁₂)cycloalkyl, and a (C₅-C₇)heterocycloalkyl, wherein moiety A is optionally substituted with 1 to R₂ groups.

Alternatively, A is selected from the group consisting of a (C₁-C₆)alkyl, a (C₃-C₁₂)cycloalkyl, and a (C₅-C₇)heterocycloalkyl, wherein moiety A is optionally substituted with 1 to 4 R₂ groups. In another alternative, A is selected from the group consisting of a (C₁-C₆)alkyl, a (C₃-C₁₂)cycloalkyl, and a (C₅-C₇)heterocycloalkyl. Further, A is ethyl or cyclohexyl.

R₁ is selected from the group consisting of —OH, a halogen, —CN, a (C₁-C₄) alkoxy, —C(O)(C₁-C₄)alkyl, —C(O)O(C₁-C₄)alkyl, -OC(O)(C₁-C₄ alkyl), —C(O)NR₃R₄, —NR₅C(═O)R₆, a (C₁-C₆)alkyl, a (C₂-C₆)alkenyl, a (C₃-C₁₂)cycloalkyl, and a (C₅-C₇)heterocycloalkyl.

Alternatively, R₁ is selected from the group consisting of —OH, a halogen, a (C₁-C₄) alkoxy, —C(O)(C₁-C₄)alkyl, —C(O)O(C₁-C₄)alkyl, —OC(O)(C₁-C₄ alkyl) and a (C₁-C₆)alkyl. Further, R₁ is selected from the group consisting of —OH, a halogen, (C₁-C₄) alkoxy, and a (C₁-C₆)alkyl. Alternatively, R₁ is selected from the group consisting of a halogen and a (C₁-C₆)alkyl. In another alternative, R₁ is selected from the group consisting of —F, —Cl, —Br, or —I.

R₂ is a (C₁-C₆)alkyl, a (C₂-C₆)alkenyl, a halo(C₁-C₆)alkoxy, a halo(C₁-C₆)alkyl, a hydroxy(C₁-C₆)alkyl, a (C₁-C₆)alkoxy(C₁-C₆)alkyl, a (C₃-C₁₂) cycloalkyl, a —(C₁-C₆)alkylene-(C₃-C₁₂)cycloalkyl, a (C₃-C₁₂) heterocycloalkyl, a —(C₁-C₆)alkylene-(C₃-C₁₂)heterocycloalkyl, a (C₁-C₆)alkoxy, —C(O)(C₁-C₆ alkyl), —C(O)O(C₁-C₆ alkyl), —OC(O)(C₁-C₆ alkyl), —C(O)NR₇R₈, —NR₉C(═O)R₁₀, —NR₁₁R₁₂, a halogen, an oxo, or —OH.

Alternatively, R₂ is a (C₁-C₆)alkyl, a halo(C₁-C₆)alkoxy, a halo(C₁-C₆)alkyl, a hydroxy(C₁-C₆)alkyl, a (C₁-C₆)alkoxy(C₁-C₆)alkyl, a (C₁-C₆)alkoxy, —C(O)(C₁-C₆ alkyl), —C(O)O(C₁-C₆ alkyl), —OC(O)(C₁-C₆ alkyl), a halogen, an oxo, or —OH. Further, R₂ is a (C₁-C₆)alkyl, a halo(C₁-C₆)alkoxy, a halo(C₁-C₆)alkyl, a hydroxy(C₁-C₆)alkyl, a (C₁-C₆)alkoxy(C₁-C₆)alkyl, a (C₁-C₆)alkoxy, a halogen, an oxo, or —OH.

R₃ is H or a (C₁-C₄)alkyl. Alternatively, R₃ is H, methyl, ethyl, propyl, iso-propyl, butyl, iso-butyl, or tert-butyl.

R₄ is H or a (C₁-C₄)alkyl. Alternatively, R₄ is H, methyl, ethyl, propyl, iso-propyl, butyl, iso-butyl, or tert-butyl.

R₅ is H or a (C₁-C₄)alkyl. Alternatively, R₅ is H, methyl, ethyl, propyl, iso-propyl, butyl, iso-butyl, or tert-butyl.

R₆ is H or a (C₁-C₄)alkyl. Alternatively, R₆ is H, methyl, ethyl, propyl, iso-propyl, butyl, iso-butyl, or tert-butyl.

R₇ is H or a (C₁-C₄)alkyl. Alternatively, R₇ is H, methyl, ethyl, propyl, iso-propyl, butyl, iso-butyl, or tert-butyl.

R₈ is H or a (C₁-C₄)alkyl. Alternatively, R₈ is H, methyl, ethyl, propyl, iso-propyl, butyl, iso-butyl, or tert-butyl.

R₉ is H or a (C₁-C₄)alkyl. Alternatively, R₉ is H, methyl, ethyl, propyl, iso-propyl, butyl, iso-butyl, or tert-butyl.

R₁₀ is H or a (C₁-C₄)alkyl. Alternatively, R₁₀ is H, methyl, ethyl, propyl, iso-propyl, butyl, iso-butyl, or tert-butyl.

R₁₁ is H or a (C₁-C₄)alkyl. Alternatively, R₁₁ is H, methyl, ethyl, propyl, iso-propyl, butyl, iso-butyl, or tert-butyl.

R₁₂ is H or a (C₁-C₄)alkyl. Alternatively, R₁₂ is H, methyl, ethyl, propyl, iso-propyl, butyl, iso-butyl, or tert-butyl.

R₂₀ is —H, —OH, a (C₁-C₃) alkyl, a (C₃-C₁₂)cycloalkyl, or a (C₅-C₇)heterocycloalkyl. Alternatively, R₂₀ is H or a (C₁-C₃)alkyl. Further, R₂₀ is H, methyl, ethyl, propyl, or iso-propyl.

R₃₀ is —H, —OH, a (C₁-C₃)alkyl, a (C₃-C₁₂)cycloalkyl, or a (C₅-C₇)heterocycloalkyl. Alternatively, R₃₀ is H or a (C₁-C₃)alkyl. Further, R₃₀ is H, methyl, ethyl, propyl, or iso-propyl.

R₄₀, for each occurence independently, is —H, —OH, a (C₁-C₃)alkyl, a (C₃-C₁₂)cycloalkyl, or a (C₅-C₇)heterocycloalkyl. R₄₀ is H or a (C₁-C₃)alkyl. Further, R₄₀ is H, methyl, ethyl, propyl, or iso-propyl.

m is 0, 1, 2, 3, or 4. Alternatively, m is 0, 1, or 2. Further, m is 1 or 2. Alternatively, m is 1.

n is 0, 1, 2, 3, or 4. Alternatively, n is 0, 1, or 2. Further, n is 0 or 1. Alternatively, n is 1.

p is 0, 1, 2, 3 or 4. Alternatively, p is 0, 1, or 2. Further, p is 0 or 1.

q is 0, 1, 2, 3 or 4. Alternatively, q is 0, 1, or 2. Further, q is 0 or 1.

A description of example embodiments of the invention follows.

A first embodiment of the present invention is directed to a compound of Structural Formula (IX):

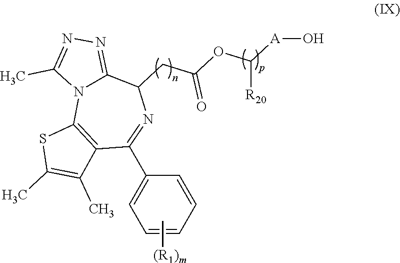

or a pharmaceutically acceptable salt thereof, wherein:

A is selected from the group consisting of a (C₁-C₆)alkyl, a (C₂-C₆)alkenyl, a (C₂-C₆)alkynyl, a (C₃-C₁₂)cycloalkyl, and a (C₅-C₇)heterocycloalkyl, wherein moiety A is optionally substituted with 1 to 4 R₂ groups;

R₂₀, for each occurence independently, is -H, —OH, a (C₁-C₃) alkyl, a (C₃-C₁₂)cycloalkyl, or a (C₅-C₇)heterocycloalkyl;

R₁ for each occurence independently is selected from the group consisting of —OH, a halogen, —CN, a (C₁-C₄) alkoxy, —C(O)(C₁-C₄)alkyl, —C(O)O(C₁-C₄)alkyl, —OC(O)(C₁-C₄ alkyl), —C(O)NR₃R₄, —NR₅C(═O)R₆, a (C₁-C₆)alkyl, a (C₂-C₆)alkenyl, a (C₃-C₁₂)cycloalkyl, and a (C₅-C₇)heterocycloalkyl;

R₂ for each occurence independently is a (C₁-C₆)alkyl, a (C₂-C₆)alkenyl, a halo(C₁-C₆)alkoxy, a halo(C₁-C₆)alkyl, a hydroxy(C₁-C₆)alkyl, a (C₁-C₆)alkoxy(C₁-C₆)alkyl, a (C₃-C₁₂) cycloalkyl, a —(C₁-C₆)alkylene-(C₃-C₁₂)cycloalkyl, a (C₃-C₁₂) heterocycloalkyl, a —(C₁-C₆)alkylene-(C₃-C₁₂)heterocycloalkyl, a (C₁-C₆)alkoxy, —C(O)(C₁-C₆ alkyl), —C(O)O(C₁-C₆ alkyl), —OC(O)(C₁-C₆ alkyl), —C(O)NR₇R₈, —NR₉C(═O)R₁₀, —NR₁₁R₁₂, a halogen, an oxo, or —OH;

R₃, R₄, R₅, R₆, R₇, R₈, R₉, R₁₀, R₁₁, and R₁₂ are each independently H or a (C₁-C₄)alkyl; and

each m, n and p is independently 0, 1, 2, 3, or 4.

In a first aspect of the first embodiment: A is a (C₁-C₆)alkyl, a (C₃-C₁₂)cycloalkyl, or a (C₅-C₇)heterocycloalkyl.

In a second aspect of the first embodiment: A is ethyl or cyclohexyl.

In a third aspect of the first embodiment: R₂ is —OH or a (C₁-C₆)alkyl. In a particular example of the third aspect, the remaining variables are as set forth in the first or second aspect of the first embodiment.

In a fourth aspect of the first embodiment: R₂ is —OH or methyl. In a particular example of the third aspect, the remaining variables are as set forth in the first or second aspect of the first embodiment.

In a fifth aspect of the first embodiment: R₁ is —F, —Cl, —Br, or —I. In a particular example of the fifth aspect, the remaining variables are as in the first, second, third or fourth aspect of the first embodiment or any of the particular examples of the third or fourth aspect.

In a sixth aspect of the first embodiment: R₂₀ is H or a (C₁-C₃)alkyl. In a particular example of the sixth aspect, the remaining variables are as in the first, second, third, fourth or fifth aspect of the first embodiment or any of the particular examples of the third, fourth or fifth aspect.

In a seventh aspect of the first embodiment: p is 0. In a particular example of the seventh aspect, the remaining variables are as in the first, second, third, fourth, fifth or sixth aspect of the first embodiment or any of the particular examples of the third, fourth or fifth or sixth aspect.

In an eighth aspect of the first embodiment: m is 1. In a particular example of the eighth aspect, the remaining variables are as in the first, second, third, fourth, fifth, sixth or seventh aspect of the first embodiment or any of the particular examples of the third, fourth, fifth, sixth or seventh aspect.

In a ninth aspect of the first embodiment: n is 1. In a particular example of the ninth aspect, the remaining variables are as in the first, second, third, fourth, fifth, sixth, seventh or eighth aspect of the first embodiment or any of the particular examples of the third, fourth, fifth, sixth, seventh or eighth aspect.

In a second embodiment, the present invention is directed to a compound of Structural Formula (X):

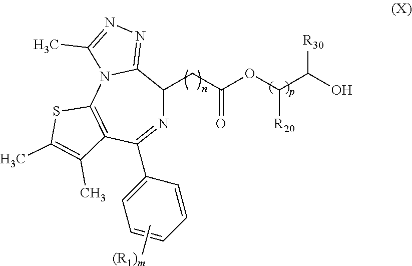

or a pharmaceutically acceptable salt thereof, wherein:

R₁ for each occurence independently is selected from the group consisting of —OH, a halogen, —CN, a (C₁-C₄) alkoxy, —C(O)(C₁-C₄)alkyl, —C(O)O(C₁-C₄)alkyl, —OC(O)(C₁-C₄ alkyl), —C(O)NR₃R₄, —NR₅C(═O)R₆, a (C₁-C₆)alkyl, a (C₂-C₆)alkenyl, a (C₃-C₁₂)cycloalkyl, and a (C₅-C₇)heterocycloalkyl;

R₃, R₄, R₅, and R₆ are each independently H or a (C₁-C₄)alkyl

R₂₀, for each occurence independently, is —H, —OH, a (C₁-C₃) alkyl, a (C₃-C₁₂)cycloalkyl, or a (C₅-C₇)heterocycloalkyl;

R₃₀, for each occurence independently, is —H, —OH, a (C₁-C₃)alkyl, a (C₃-C₁₂)cycloalkyl, or a (C₅-C₇)heterocycloalkyl; and

each m, n and p is independently 0, 1, 2, 3, or 4.

In a first aspect of the second embodiment: R₁ is —F, —Cl, —Br, or —I.

In a second aspect of second embodiment: R₂₀ is H or a (C₁-C₃)alkyl. In a particular example of the second aspect, the remaining variables are as set forth in the first aspect of the second embodiment.

In a third aspect of the second embodiment: R₃₀ is H or a (C₁-C₃)alkyl. In a particular example of the third aspect, the remaining variables are as set forth in the first or second aspect of the second embodiment or any of the particular examples of the second aspect.

In a fourth aspect of the second embodiment: p is 1. In a particular example of the fourth aspect, the remaining variables are as set forth in the first, second or third aspect of the second embodiment or any of the particular examples of the second or third aspect.

In a fifth aspect of the second embodiment: m is 1. In a particular example of the fifth aspect, the remaining variables are as set forth in the first, second, third or fourth aspect of the second embodiment or any of the particular examples of the second, third or fourth aspect.

In a sixth aspect of the second embodiment: n is 1. In a particular example of the sixth aspect, the remaining variables are as set forth in the first, second, third, fourth or fifith aspect of the second embodiment or any of the particular examples of the second, third, fourth or fifth aspect.

In a third embodiment, the present invention is directed to a compound of Structural Formula (XI):

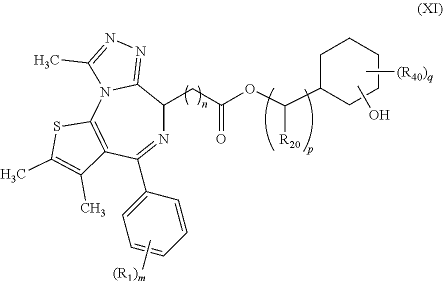

or a pharmaceutically acceptable salt thereof, wherein:

R₁ for each occurence independently is selected from the group consisting of —OH, a halogen, —CN, a (C₁-C₄) alkoxy, —C(O)(C₁-C₄)alkyl, —C(O)O(C₁-C₄)alkyl, —OC(O)(C₁-C₄ alkyl), —C(O)NR₃R₄, —NR₅C(═O)R₆, a (C₁-C₆)alkyl, a (C₂-C₆)alkenyl, a (C₃-C₁₂)cycloalkyl, and a (C₅-C₇)heterocycloalkyl;

R₃, R₄, R₅, and R₆ are each independently H or a (C₁-C₄)alkyl

R₂₀, for each occurence independently, is -H, —OH, a (C₁-C₃) alkyl, a (C₃-C₁₂)cycloalkyl, or a (C₅-C₇)heterocycloalkyl;

R₄₀, for each occurence independently, is —H, —OH, a (C₁-C₃)alkyl, a (C₃-C₁₂)cycloalkyl, or a (C₅-C₇)heterocycloalkyl; and

each q, m, n and p is independently 0, 1, 2, 3 or 4.

In one aspect of the third embodiment: R₁ is —F, —Cl, —Br, or —I.

In a second aspect of third embodiment: R₂₀ is H or a (C₁-C₃)alkyl. In a particular example of the second aspect, the remaining variables are as set forth in the first aspect of the third embodiment.

In a third aspect of the third embodiment: R₄₀ is H or a (C₁-C₃)alkyl. In a particular example of the third aspect, the remaining variables are as set forth in the first or second aspect of the third embodiment or any of the particular examples of the second aspect.

In a fourth aspect of the third embodiment: p is 0. In a particular example of the fourth aspect, the remaining variables are as set forth in the first, second or third aspect of the third embodiment or any of the particular examples of the second or third aspect.

In a fifth aspect of the third embodiment: m is 1. In a particular example of the fifth aspect, the remaining variables are as set forth in the first, second, third or fourth aspect of the third embodiment or any of the particular examples of the second, third or fourth aspect.

In a sixth aspect of the third embodiment: n is 1. In a particular example of the sixth aspect, the remaining variables are as set forth in the first, second, third, fourth or fifth aspect of the third embodiment or any of the particular examples of the second, third, fourth or fifth aspect.

In another aspect, the invention provides a compound represented by any one of the following formulae:

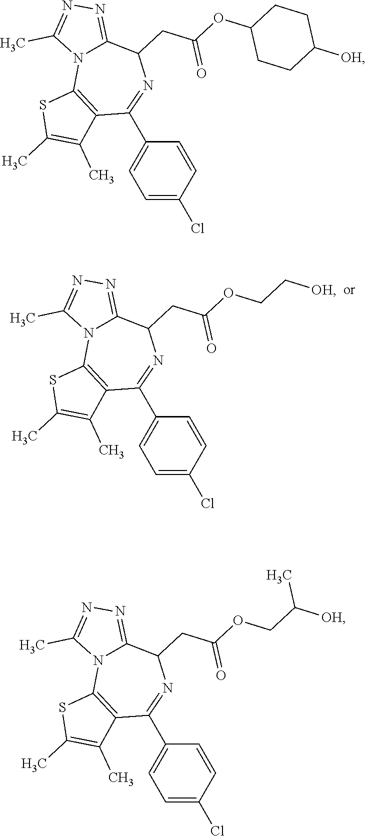 [SMILES: Cc1sc2c(c1C)C(c1ccc(Cl)cc1)=NC(CC(=O)OC1CCC(O)CC1)c1nnc(C)n1-2 ; Cc1sc2c(c1C)C(c1ccc(Cl)cc1)=NC(CC(=O)OCCO)c1nnc(C)n1-2 ; Cc1sc2c(c1C)C(c1ccc(Cl)cc1)=NC(CC(=O)OCC(C)O)c1nnc(C)n1-2]

or a pharmaceutically acceptable salt thereof.

In another aspect, the invention provides a compound represented by any one of the following formulae:

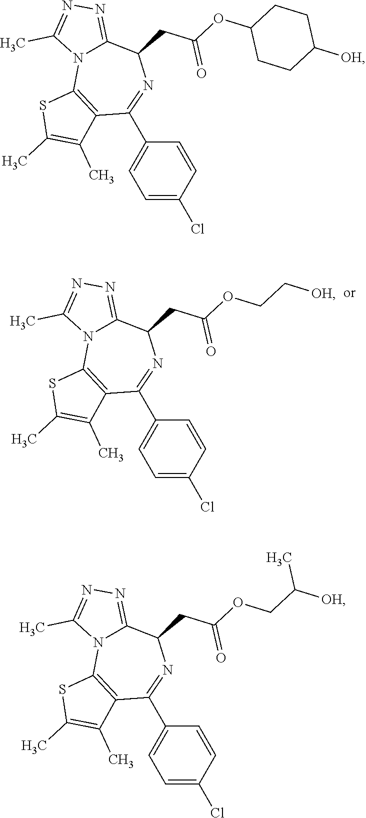 [SMILES: Cc1sc2c(c1C)C(c1ccc(Cl)cc1)=N[C@H](CC(=O)OC1CCC(O)CC1)c1nnc(C)n1-2 ; Cc1sc2c(c1C)C(c1ccc(Cl)cc1)=N[C@H](CC(=O)OCCO)c1nnc(C)n1-2 ; Cc1sc2c(c1C)C(c1ccc(Cl)cc1)=N[C@H](CC(=O)OCC(C)O)c1nnc(C)n1-2]

or a pharmaceutically acceptable salt thereof.

In another aspect, the invention provides a compound represented by any one of the following formulae:

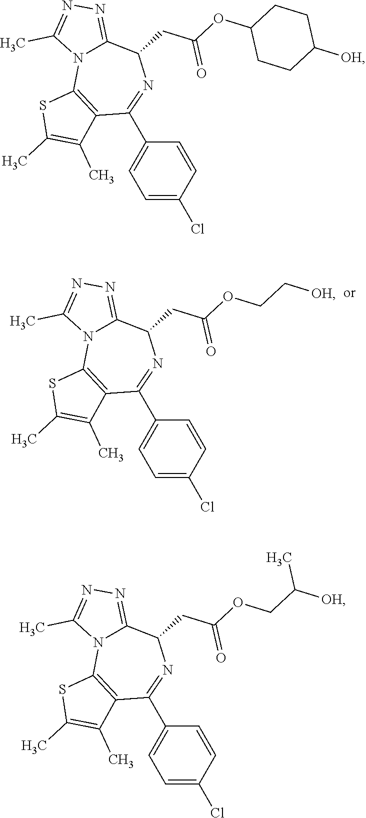 [SMILES: Cc1sc2c(c1C)C(c1ccc(Cl)cc1)=N[C@@H](CC(=O)OC1CCC(O)CC1)c1nnc(C)n1-2 ; Cc1sc2c(c1C)C(c1ccc(Cl)cc1)=N[C@@H](CC(=O)OCCO)c1nnc(C)n1-2 ; Cc1sc2c(c1C)C(c1ccc(Cl)cc1)=N[C@@H](CC(=O)OCC(C)O)c1nnc(C)n1-2]

or a pharmaceutically acceptable salt thereof.

In another aspect, the invention provides a compound represented by any one of the following formulae:

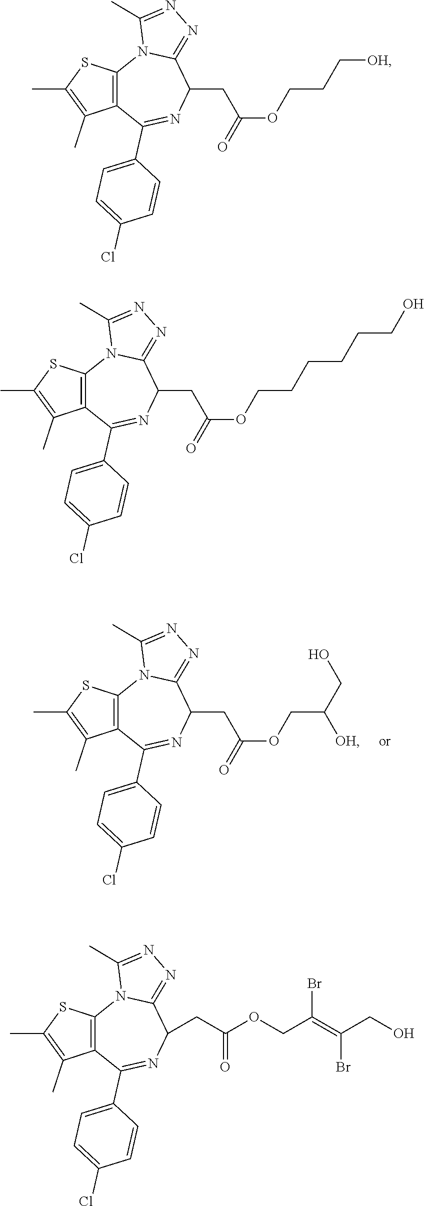 [SMILES: Cc1sc2c(c1C)C(c1ccc(Cl)cc1)=NC(CC(=O)OCCCO)c1nnc(C)n1-2 ; Cc1sc2c(c1C)C(c1ccc(Cl)cc1)=NC(CC(=O)OCCCCCCO)c1nnc(C)n1-2 ; Cc1sc2c(c1C)C(c1ccc(Cl)cc1)=NC(CC(=O)OCC(O)CO)c1nnc(C)n1-2 ; Cc1sc2c(c1C)C(c1ccc(Cl)cc1)=NC(CC(=O)OC/C(Br)=C(\Br)CO)c1nnc(C)n1-2]

or a pharmaceutically acceptable salt thereof.

In another aspect, the invention provides a compound represented by any one of the following formulae:

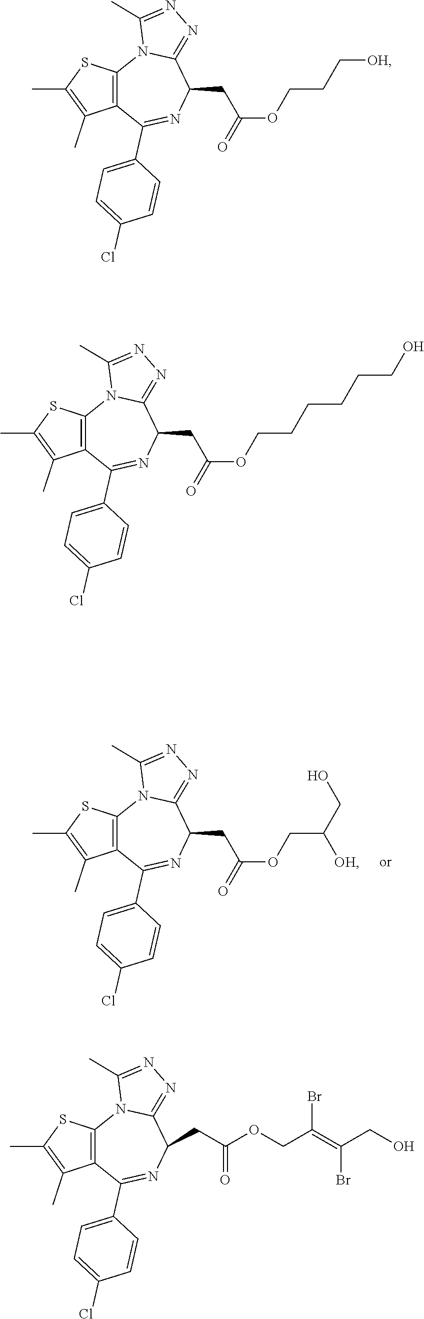 [SMILES: Cc1sc2c(c1C)C(c1ccc(Cl)cc1)=N[C@H](CC(=O)OCCCO)c1nnc(C)n1-2 ; Cc1sc2c(c1C)C(c1ccc(Cl)cc1)=N[C@H](CC(=O)OCCCCCCO)c1nnc(C)n1-2 ; Cc1sc2c(c1C)C(c1ccc(Cl)cc1)=N[C@H](CC(=O)OCC(O)CO)c1nnc(C)n1-2 ; Cc1sc2c(c1C)C(c1ccc(Cl)cc1)=N[C@H](CC(=O)OC/C(Br)=C(\Br)CO)c1nnc(C)n1-2]

or a pharmaceutically acceptable salt thereof.

In another aspect, the invention provides a compound represented by any one of the following formulae:

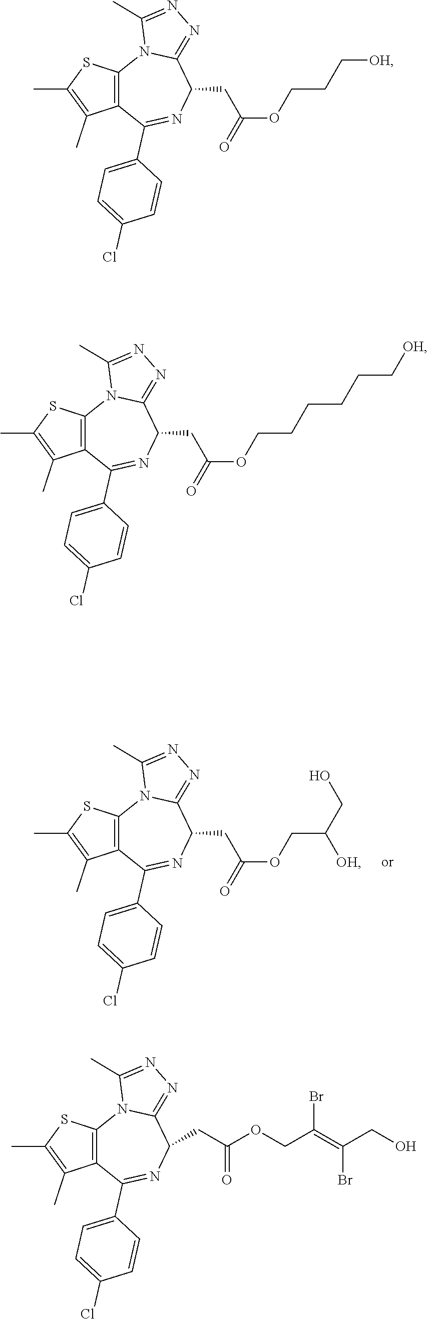 [SMILES: Cc1sc2c(c1C)C(c1ccc(Cl)cc1)=N[C@@H](CC(=O)OCCCO)c1nnc(C)n1-2 ; Cc1sc2c(c1C)C(c1ccc(Cl)cc1)=N[C@@H](CC(=O)OCCCCCCO)c1nnc(C)n1-2 ; Cc1sc2c(c1C)C(c1ccc(Cl)cc1)=N[C@@H](CC(=O)OCC(O)CO)c1nnc(C)n1-2 ; Cc1sc2c(c1C)C(c1ccc(Cl)cc1)=N[C@@H](CC(=O)OC/C(Br)=C(\Br)CO)c1nnc(C)n1-2]

or a pharmaceutically acceptable salt thereof.

Further examples of BET inhibitors suitable for using with the methods disclosed herein include the compounds and compositions disclosed in WO 2011/054843 (Glaxosmithkline), WO 2009/084693 (Mitsubishi Tanabe Pharma Corporation), WO 2012/075383 (Constellation Pharmaceuticals, Inc.), WO 2011/054553 (Glaxosmithkline), WO 2011/054841 (Glaxosmithkline), WO 2011/054844 (Glaxosmithkline), WO 2011/054845 (Glaxosmithkline), WO 2011/054846 (Glaxosmithkline), WO 2011/054848 (Glaxosmithkline), WO 2011/161031 (Glaxosmithkline), US2015/0148337 (Constellation Pharmaceuticals, Inc.), US2014/0371206 (Constellation Pharmaceuticals, Inc.), US2014/0296243 (Constellation Pharmaceuticals, Inc.), US2014/0135316 (Constellation Pharmaceuticals, Inc.), US2014/0005169 (Constellation Pharmaceuticals, Inc.), US2012/0157428 (Constellation Pharmaceuticals, Inc.), and U.S. Pat No. 8,796,261 (Constellation Pharmaceuticals, Inc.). The relevant teachings of each of these documents are incorporated herein by reference.

Modes of Administration

The bromodomain inhibitors (e.g., TEN-010) for use in the methods or compositions of the invention can be formulated for parenteral, oral, transdermal, sublingual, buccal, rectal, intranasal, intrabronchial or intrapulmonary administration.

For parenteral administration, the compounds for use in the methods or compositions of the invention can be formulated for injection or infusion, for example, intravenous, intramuscular or subcutaneous injection or infusion, or for administration in a bolus dose and/or infusion (e.g., continuous infusion). Suspensions, solutions or emulsions in an oily or aqueous vehicle, optionally containing other formulatory agents such as suspending, stabilizing and/or dispersing agents can be used.

For oral administration the bromodomain inhibitor can be of the form of tablets or capsules prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated using suitable methods. Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the methods or compositions of the invention can be in the form of tablets or lozenges formulated in a conventional manner.

For rectal administration, the compounds for use in the methods or compositions of the invention can be in the form of suppositories.

For sublingual administration, tablets can be formulated in conventional manner.

For intranasal, intrabronchial or intrapulmonary administration, conventional formulations can be employed.

Further, the compounds for use in the methods or compositions of the invention can be formulated in a sustained release preparation. For example, the compounds can be formulated with a suitable polymer or hydrophobic material which provides sustained and/or controlled release properties to the active agent compound. As such, the compounds for use in the method of the invention can be administered in the form of microparticles, for example, by injection or in the form of wafers or discs by implantation. Various methods of formulating controlled release drug preparations are known in the art.

Administration of a bromodomain inhibitor, or pharmaceutically acceptable salt thereof, disclosed herein useful to practice the methods described herein, can be continuous, hourly, four times daily, three time daily, twice daily, once daily, once every other day, twice weekly, once weekly, once every two weeks, once a month, or once every two months, or longer, or some other intermittent dosing regimen. In a particular embodiment, the bromodomain inhibitor is administered in cycles, as described herein.

Examples of administration of a bromodomain inhibitor, or pharmaceutical salt thereof, of the invention include peripheral administration. Examples of peripheral administration include oral, subcutaneous, intraperitoneal, intramuscular, intravenous, rectal, transdermal, or intranasal forms of administration.

As used herein, peripheral administration includes all forms of administration of a bromodomain inhibitor or a composition comprising a bromodomain inhibitor disclosed herein which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

Pharmaceutical Composition

The bromodomain inhibitors disclosed herein can be incorporated into pharmaceutical compositions suitable for administration. Such compositions typically comprise the bromodomain inhibitor (e.g., TEN-010) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

A pharmaceutical composition of the invention is formulated to be compatible with its intended route of administration. As described herein, examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., TEN-010) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the bromodomain inhibitor can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the bromodomain inhibitors are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Suitable doses per administration for a bromodomain inhibitor include doses of about or greater than about 250 ng/kg, about 500 ng/kg, about 750 ng/kg, about 1 ug/kg, about 10 ug/kg, about 20 ug/kg, about 30 ug/kg, about 40 ug/kg, about 50 ug/kg, about 60 ug/kg, about 70 ug/kg, about 80 ug/kg, about 90 ug/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, or about 2.0 mg/kg. Each suitable dose can be administered over a period time deemed appropriate by a skilled practitioner. In one example, each suitable dose of TEN-010 can be administered in a single injection, at about 0.45 mg/kg, or about 0.65 mg/kg. In other embodiments, each suitable dose can be administered (e.g., infused) over a period of time deemed appropriate by a skilled professional.

Combination Therapy

The bromodomain inhibitors (e.g., TEN-010) disclosed herein can be used for treating NMC in combination with a second amount of an anti-cancer agent (sometime referred to herein as a “second agent”), e.g., chemotherapeutic agents or HDAC inhibitors. Such combination administration can be by means of a single dosage form which includes a bromodomain inhibitor and the second agent, such single dosage form including a tablet, capsule, spray, inhalation powder, injectable liquid or the like. Combination administration can comprise a further second agent (e.g., chemotherapeutic agent or HDAC inhibitor) in addition to the single dosage form. Alternatively, combination administration can be by means of administration of two different dosage forms, with one dosage form containing a bromodomain inhibitor, and the other dosage form including a second amount of an anti-cancer agent. In this instance, the dosage forms may be the same or different. Without wishing to limit combination therapies, the following exemplifies certain combination therapies which may be employed. It is understood that additional anti-cancer agents beyond the required second amount of an anti-cancer agent can be employed in the method described herein.

The second amount of the anti-cancer agent (sometimes referred to herein as the second agent) can be administered before, simultaneously with, or after the administration of a bromodomain inhibitor. Accordingly, a bromodomain inhibitor and a second agent can be administered together in a single formulation or can be administered in separate formulations, e.g., either simultaneously or sequentially, or both. For example, if a bromodomain inhibitor and a second agent are administered sequentially in separate compositions, the bromodomain inhibitor can be administered before or after the anti-cancer agent. The duration of time between the administration of a bromodomain inhibitor and the second amount of the anti-cancer agent will depend on the nature of the anti-cancer agent. In certain embodiments, the bromodomain inhibitor can precede or follow a chemotherapeutic agent immediately, or after some duration of time deemed to be appropriate by a skilled practitioner.

In addition, the bromodomain inhibitor and the second amount of the anti-cancer agent may or may not be administered on similar dosing schedules. For example, the brmodomain inhibitor and the anti-cancer agent may have different half-lives and/or act on different time-scales such that the bromodomain inhibitor is administered with greater frequency than the anti-cancer agent or vice-versa. For example, the bromodomain inhibitor and the anti-cancer agent can be administered together (e.g., in a single dosage or sequentially) on one day, followed by administration of only the bromodomain inhibitor for a set number of subsequent days. The number of days in between administration of therapeutic agents can be appropriately determined according to the safety and pharmacodynamics of each drug. Either the bromodomain inhibitor or the anti-cancer agent can be administered acutely or chronically.

Suitable doses per administration of a bromodomain inhibitor have been described herein. An effective amount of the second active agent (e.g., chemotherapeutic agent or HDAC inhibitor) will depend on the age, gender, and weight of the patient, the current medical condition of the patient, and the nature of the NMC being treated. Those of skill in the art will be able to determine appropriate dosages depending on these and other factors. Suitable doses per administration for a second amount of an anti-cancer agent in a combination therapy can be determined based on the recommended dosing found on the label, as appropriate by a skilled medical professional.

### EXAMPLES OF THE INVENTION

Compound TEN-010: The Compound TEN-010 used in the following examples and disclosed herein has the following structural formula:

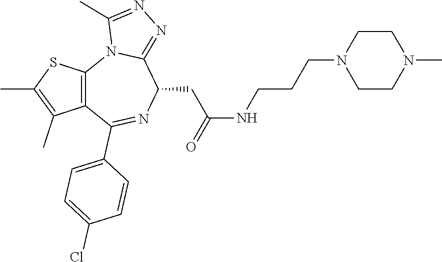

CD11b Expression Levels in NMC Patient is Indicative of Disease Activity

The present study was designed to evaluate whether the BET bromodomain inhibitor TEN-010 could have potential to be of benefit in solid tumor oncology indications. As demonstrated herein, the levels of CD11b expressed on the surface of monocytes serve as a marker of responsiveness in TEN-010 NMC therapy.

The clinical studies disclosed herein were performed in compliance with Good Clinical Practice (GCP), the ethical principles stated in the Declaration of Helsinki, and other applicable regulatory requirements.

Materials and Methods

Study Population

Patients aged 18 years or older with histologically confirmed advanced solid tumors with progressive disease requiring therapy were enrolled in the study. In particular, patients with histologically confirmed advanced solid malignancy with progressive disease, NMC, or advanced aggressive diffuse large B cell lymphoma (DLBCL) were enrolled in the study. Patients with hemotologic malignancies were not enrolled in the study.

Administration of TEN-010

TEN-010 was formulated as a sterile, preserved isotonic solution for subcutaneous (SC) administration. A dose of 0.45 mg/kg was administered on Days 1 through 21 (“ON segment”) of each cycle without interruption, followed by a 7-day dose-free interval (“OFF segment”) in a 28-day treatment cycle. Injections were rotated amongst several sites including bilateral upper arms and thighs and mid and lower abdomen and buttocks.

Sample Collection and Assay for CD11b Expression Levels

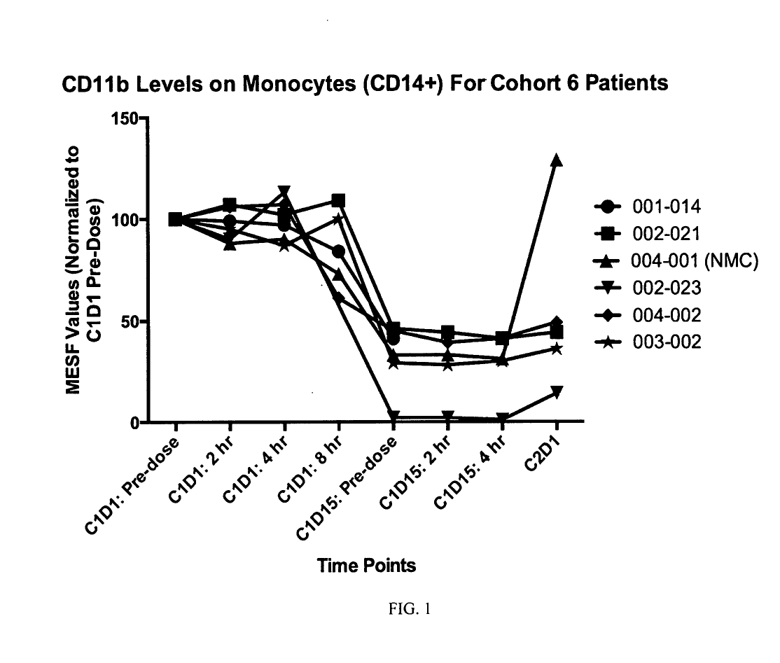

Whole blood specimens were collected at specified time points (e.g., see FIG. 1) in sodium heparin vacutainers. Briefly, two 12×75 mm test tubes per donor were labeled with specimen ID and appropriate cocktail name (see Table 2 below). 100 μl of sodium heparin anticoagulated whole blood was pipetted into the test tubes. The appropriate titrated volume of antibody cocktails was pipetted into the correspondingly labeled tubes. The appropriate titrated amount of CD14 PerCP, was added to all tubes to identify CD14 positive monocytes. The tubes were vortexed and were allowed to incubate for 30 minutes at room temperature in the dark. The red blood cells were lysed by adding 4 ml of an ammonium chloride-based whole blood lysing reagent to each tube. The tubes were capped and inverted to mix well prior to incubating in the dark at room temperature for 5 minutes. After the incubation, the tubes were centrifuged at 400 RCF for 5 minutes, the supernatant decanted and the tubes rack-raked to disperse cell pellet. The cells were washed with 2 ml of PBS with 1% BSA and centrifuged. To detect biotin conjugated CD45 RO antibody, the appropriate titrated amount of SA-BV605 (streptavidin (SA) conjugated to brilliant violet (BV) 605) was added to each tube and tubes were vortexed. After a 20 minute incubation in the dark at room temperature, the cells were washed with 2 ml of PBS with 1% BSA and centrifuged. The supernatant was decanted and the cells were rack-raked to disperse the cell pellet. Each tube received 500 μl of 1% paraformaldehyde and stored at 2-8 C until acquisition on the day of preparation. The tubes were acquired on a Becton Dickinson (BD) FACSCanto™ II flow cytometer with appropriate instrument settings, acquiring approximately 250,000 total events per tube.

TABLE 2
Flow cytometry labeling mix

| | Content of Cocktail Mixture |
|---|---|
| Cocktail #1 | MsIgG1 FITC, MsIgG2a phycoerythrin (PE), MsIgG1 |
| (control to | Allophycocyanin (APC), CD4 Alexa Fluor ® 700 (AF700), |
| determine | MsIgG1 mFluor ™ Violet 450 (V450), CD3 Violet 500 |
| background | (V500), and CD45RO Biotin-SA BV605 |
| fluorescence) | |
| Cocktail #2 | CD127 FITC, E-Selectin(CD62E) PE, MAC-1 (CD11b) |
| | APC, CD4 AF700, CD25 V450, CD3 V500 and CD45RO |
| | Biotin-SA BV605 |

All labeled antibody reagents for flow cytometric assay were purchased from Becton Dickinson; E-Selectin (CD62E) PE and CD45RO Biotin were purchased from Biolegend.

Other reagents used is the study include PBS with 1% BSA, ammonium chloride-based whole blood lysing reagent, 1% paraformaldehyde solution, and Quantum MESF fluorescein isothiocyanate (FITC), phycoerythrin (PE), Allophycocyanin (APC) Calibration Beads.

### Assay for Measuring LDH Levels

LDH levels were measured using standard protocols, using a chemistry analyzer, e.g., Beckman Coulter. See, e.g., Lactate OSR6193 procedure published March 2012 (webcache.googleuserconent.com/search?q=cache:iyYi7vCetH4J:https://www.beckmancoulter.com/wsrportal/techdocs%3Fdocname%3D/cis/BAOSR6x93%2525%2525/EN_LACTATE_BAOSR6x93_US.doc+&cd=2&hl=en&ct=clnk&gl=us), incorporated by reference in its entirety.

Data Analysis

All analyses for flow cytometry were performed on WinList 7.0 (Verity Software House, Topsham, Me.) with a direct data exchange link to Microsoft® Excel 2003 or equivalent. For FIG. 1, baseline value (pre-dose at cycle 1, day 1, i.e., C1D1) was set at an arbitrary MESF value (e.g., 100) for each patient; all subsequent values obtained from the study were normalized to the baseline value. For FIG. 2, unnormalized MESF values are shown. The MESF values obtained for the pre-dose, 2, 4 and 8 hour time points on C1D1 were averaged and have been displayed as a single value for C1D1. The MESF values obtained for the pre-dose, 2 and 4 hour time points on C 1D15 were averaged and have been displayed as a single value for C1D15. Patients that did not have C1D1 or C1D15 data available are not shown.

Results

As described herein, CD11b levels on CD14+ monocytes were measured in all 6 patients in the present study. FIG. 1 shows a representative data set collected for each patient at the indicated timepoints. CD11b levels in all patients decreased by at least 50% of the baseline value (pre-dose at cycle 1 day 1—C1D1) by cycle 1 day 15 (C1D15). At the completion of one cycle (e.g., 21 days of on-drug segment followed by 7 days of off-drug segment), and at the start of the second cycle (C2D1), CD11b levels held steady in all patients except patient 004-001, who suffered from NMC (FIG. 1). The CD11b levels in this patient dramatically increased following the off-drug segment, suggesting that TEN-010 was not effective in this patient by C2D1. Patient 004-001 died shortly thereafter.

In conjunction with the measurement of CD11b expression levels, lactate dehydrogenase (LDH) levels were also measured along similar timepoints. LDH is a known clinical biomarker for cancer progression, and is routinely measured as part of cancer diagnosis and disease progression. Notably, as shown in FIG. 2C, CD11b levels in the NMC patient tracked with LDH levels, validating CD11b levels as a marker for responsiveness in the NMC patient. In contrast, CD11b levels were independent of LDH levels in non-NMC patients. In fact, for non-NMC Patent 002-021 (FIG. 2B), LDH levels remained constant despite a significant rise in CD11b levels.

Taken together, these results suggest, in part, that CD11b levels can be used to monitor NMC responsiveness to a bromodomain inhibitor therapy. Further, while not wishing to be bound by any theory, monitoring CD11b levels on monocytes enables one to follow NMC disease activity. Accordingly, CD11b levels can be measured to determine whether an NMC patient will require more or less bromodomain inhibitor in subsequent cycle(s) of treatment, or whether an NMC patient will require an earlier or delayed commencement of a subsequent cycle of bromodomain inhibitor treatment, or any combination thereof.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a patient suffering from nuclear protein in testis (NUT) midline carcinoma (NMC), comprising:
administering an effective amount of a bromodomain inhibitor to the patient in a current cycle of a treatment regimen having multiple cycles, each cycle including an on-drug and an off-drug segment,
wherein the patient exhibits a CD11b expression reduction of less than about 50% relative to a baseline level, and wherein the CD11b expression is measured during the current cycle or a prior cycle.

2. The method of claim 1, wherein the CD11b expression is measured during the off-drug segment of the prior cycle.

3. The method of claim 1, wherein the CD11b expression is measured during the on-drug segment of the current cycle.

4. A method of monitoring a treatment response in a patient suffering from nuclear protein in testis (NUT) midline carcinoma (NMC), comprising:
a) administering a predetermined amount of a bromodomain inhibitor to the patient using a treatment regimen having multiple cycles, each cycle comprising an on-drug and an off-drug segment; and
b) quantifying a CD11b expression level in a sample collected from the patient;
wherein a CD11b expression reduction of about 50% or more relative to a baseline level indicates a positive response to the treatment regimen.

5. The method of claim 4, wherein the CD11b expression level is quantified during the off-drug segment of at least one cycle.

6. A method of determining a treatment regimen in a patient suffering from nuclear protein in testis (NUT) midline carcinoma (NMC), comprising:
a) administering a predetermined amount of a bromodomain inhibitor to the patient in a first cycle of a treatment regimen having multiple cycles, each cycle including an on-drug and an off-drug segment;
b) quantifying a CD11b expression level in a sample collected from the patient during the first cycle; and
c) determining whether to modify the first cycle or a subsequent cycle of the treatment regimen, wherein a CD11b expression reduction of less than about 50% relative to a baseline level indicates that the first cycle or the subsequent cycle should be modified, thereby determining the treatment regimen in a patient suffering from NMC.

7. The method of claim 6, wherein the first cycle or the subsequent cycle is modified by increasing the length of the on-drug segment, decreasing the length of the off-drug segment, increasing the predetermined amount of the bromodomain inhibitor, or a combination thereof.

8. The method of claim 6, wherein the CD11b expression level is quantified during the off-drug segment of the first or subsequent cycles.

9. The method of claim 6, wherein the CD11b expression level is quantified during the on-drug segment of the first cycle.

10. The method of any one of claims 1-9, wherein the bromodomain inhibitor is represented by Structural Formula IV: 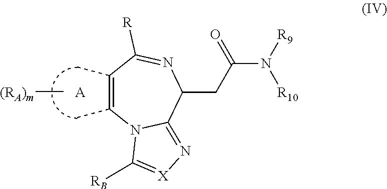
or a pharmaceutically acceptable salt thereof, wherein:
X is N or CR₃;
R₃ is selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₁₀)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₁₀)heteroaryl is optionally and independently substituted with 1 to 4 substituents;
R_{B} is H, —(C₁-C₄)alkyl, —(C₁-C₄)alkylene-O—(C₁-C₄)alkyl, or —COO—R₄, wherein each —(C₁-C₄)alkyl and —(C₁-C₄)alkylene-O—(C₁-C₄)alkyl is optionally substituted with 1 to 4 substituents independently selected from the group consisting of —F, —Cl, —Br, —OH, and —NR₅R₆;
ring A is aryl or heteroaryl;
each R_{A} is independently H, —(C₁-C₄)alkyl, —(C₃-C₈)cycloalkyl, —(C₅-C₇) heterocycloalkyl, —(C₆-C₁₀)aryl, or —(C₅-C₁₀)heteroaryl, wherein each —(C₁-C₄)alkyl, —(C₃-C₈)cycloalkyl, —(C₅-C₇) heterocycloalkyl, —(C₆-C₁₀)aryl, and —(C₅-C₁₀)heteroaryl is optionally and independently substituted with 1 to 4 substituents; or any two R_{A} together with the atoms to which each is bound form a fused aryl or heteroaryl group;
R is —(C₁-C₄)alkyl, —(C₃-C₈)cycloalkyl, —(C₅-C₇)heterocycloalkyl, —(C₆-C₁₀)aryl, or —(C₅-C₁₀)heteroaryl, wherein each is optionally and independently substituted with 1 to 4 substituents;
R₄, R₅, and R₆ are each independently selected from the group consisting of: H, —(C₁-C₄)alkyl, —(C₃-C₈)cycloalkyl, —(C₅-C₇)heterocycloalkyl, —(C₆-C₁₀)aryl, and —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents;
R₉ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl, —(C₀-C₆)alkylene-heteroaryl, and —N═CR₁₁R₁₂, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;
R₁₀ is selected from the group consisting of: H, —(C₁-C₆)alkyl, —(C₀-C₆)alkylene-cycloalkyl, —(C₀-C₆)alkylene-heterocycloalkyl, —(C₀-C₆)alkylene-aryl; and —(C₀-C₆)alkylene-heteroaryl, wherein each —(C₁-C₆)alkyl and —(C₀-C₆)alkylene- is optionally and independently substituted with 1 to 4 substituents and each -cycloalkyl, -heterocycloalkyl, -aryl, and -heteroaryl is optionally and independently substituted with 1 to 4 substituents;
R₉ and R₁₀ are taken together with the nitrogen atom to which they are bound form a 4-10-membered ring;
R₁₁ is H, —(C₁-C₄)alkyl, or —(C₁-C₄)alkylene-O—(C₁-C₄)alkyl, wherein each —(C₁-C₄)alkyl and —(C₁-C₄)alkylene-O—(C₁-C₄)alkyl is optionally substituted with 1 to 3 substituents selected from the group consisting of: —F, —Cl, —Br, and —OH;
R₁₂ is H, —(C₁-C₄)alkyl, —(C₃-C₈)cycloalkyl, —(C₅-C₇)heterocycloalkyl, —(C₆-C₁₀)aryl, or —(C₅-C₇)heteroaryl, wherein each —(C₁-C₄)alkyl, —(C₃-C₈)cycloalkyl, —(C₅-C₇)heterocycloalkyl, —(C₆-C₁₀)aryl, and —(C₅-C₇)heteroaryl is optionally and independently substituted with 1 to 4 substituents; and
m is 0, 1, 2, or 3.

11. The method of any one of claims 1-10, wherein the bromodomain inhibitor is a compound is represented by represented by any one of the following structural formulas: 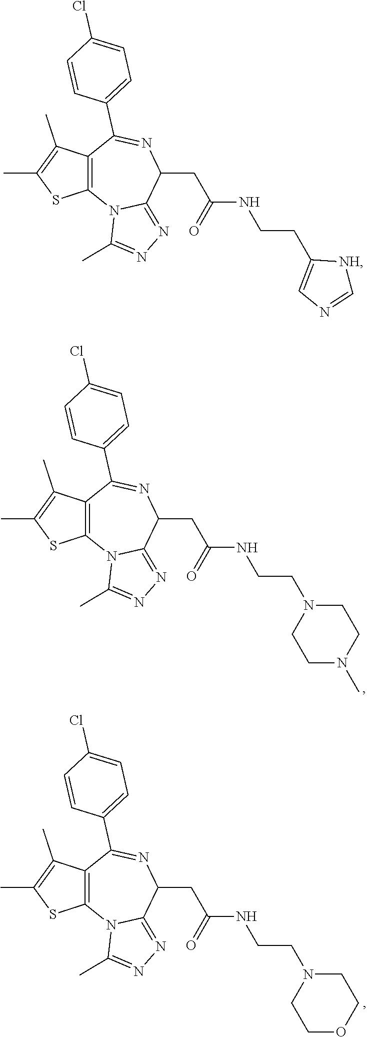 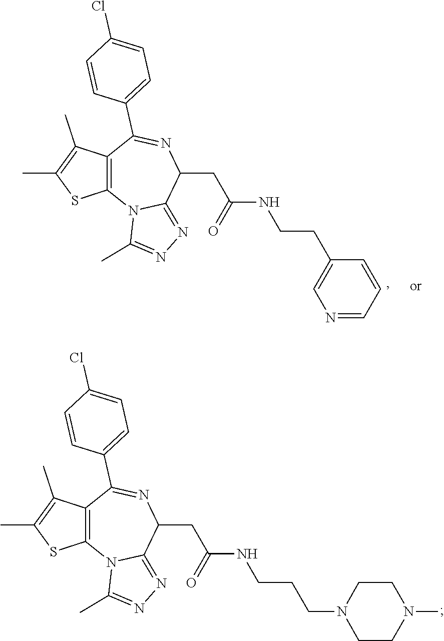
or a pharmaceutically acceptable salt thereof.

12. The method of any one of claims 1-11, wherein the bromodomain inhibitor is a compound represented by the structural formula: 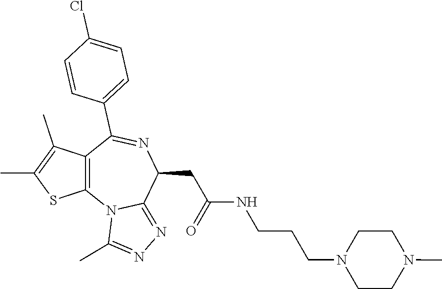
or a pharmaceutically acceptable salt thereof.

13. The method of any one of claims 1-9, wherein the bromodomain inhibitor is a compound represented by Structural Formula (IX): 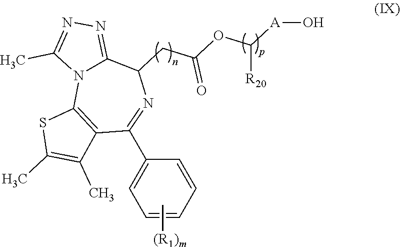
or a pharmaceutically acceptable salt thereof, wherein:
A is selected from the group consisting of a (C₁-C₆)alkyl, a (C₂-C₆)alkenyl, a (C₂-C₆)alkynyl, a (C₃-C₁₂)cycloalkyl, and a (C₅-C₇)heterocycloalkyl, wherein moiety A is optionally substituted with 1 to 4 R₂ groups;
R₂₀, for each occurence independently, is -H, —OH, a (C₁-C₃) alkyl, a (C₃-C₁₂)cycloalkyl, or a (C₅-C₇)heterocycloalkyl;
R₁ for each occurence independently is selected from the group consisting of —OH, a halogen, —CN, a (C₁-C₄) alkoxy, —C(O)(C₁-C₄)alkyl, —C(O)O(C₁-C₄)alkyl, —OC(O)(C₁-C₄ alkyl), —C(O)NR₃R₄, —NR₅C(═O)R₆, a (C₁-C₆)alkyl, a (C₂-C₆)alkenyl, a (C₃-C₁₂)cycloalkyl, and a (C₅-C₇)heterocycloalkyl;
R₂ for each occurence independently is a (C₁-C₆)alkyl, a (C₂-C₆)alkenyl, a halo(C₁-C₆)alkoxy, a halo(C₁-C₆)alkyl, a hydroxy(C₁-C₆)alkyl, a (C₁-C₆)alkoxy(C₁-C₆)alkyl, a (C₃-C₁₂) cycloalkyl, a —(C₁-C₆)alkylene-(C₃-C₁₂)cycloalkyl, a (C₃-C₁₂) heterocycloalkyl, a —(C₁-C₆)alkylene-(C₃-C₁₂)heterocycloalkyl, a (C₁-C₆)alkoxy, —C(O)(C₁-C₆ alkyl), —C(O)O(C₁-C₆ alkyl), —OC(O)(C₁-C₆ alkyl), —C(O)NR₇R₈, —NR₉C(═O)R₁₀, —NR₁₁R₁₂, a halogen, an oxo, or —OH;
R₃, R₄, R₅, R₆, R₇, R₈, R₉, R₁₀, R₁₁, and R₁₂ are each independently H or a (C₁-C₄)alkyl; and
each m, n and p is independently 0, 1, 2, 3, or 4.

14. The method of any one of the claim 1-9 or 13, wherein the bromodomain inhibitor is a compound represented by any one of the following structural formulae: 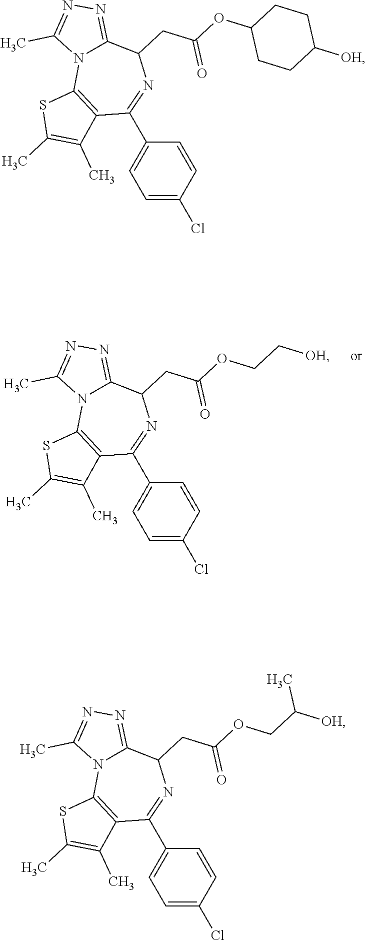 [SMILES: Cc1sc2c(c1C)C(c1ccc(Cl)cc1)=NC(CC(=O)OC1CCC(O)CC1)c1nnc(C)n1-2 ; Cc1sc2c(c1C)C(c1ccc(Cl)cc1)=NC(CC(=O)OCCO)c1nnc(C)n1-2 ; Cc1sc2c(c1C)C(c1ccc(Cl)cc1)=NC(CC(=O)OCC(C)O)c1nnc(C)n1-2]
or a pharmaceutically acceptable salt thereof.

15. The method of any one of the claim 1-9 or 13, wherein the bromodomain inhibitor is a compound represented by by any one of the following formulae: 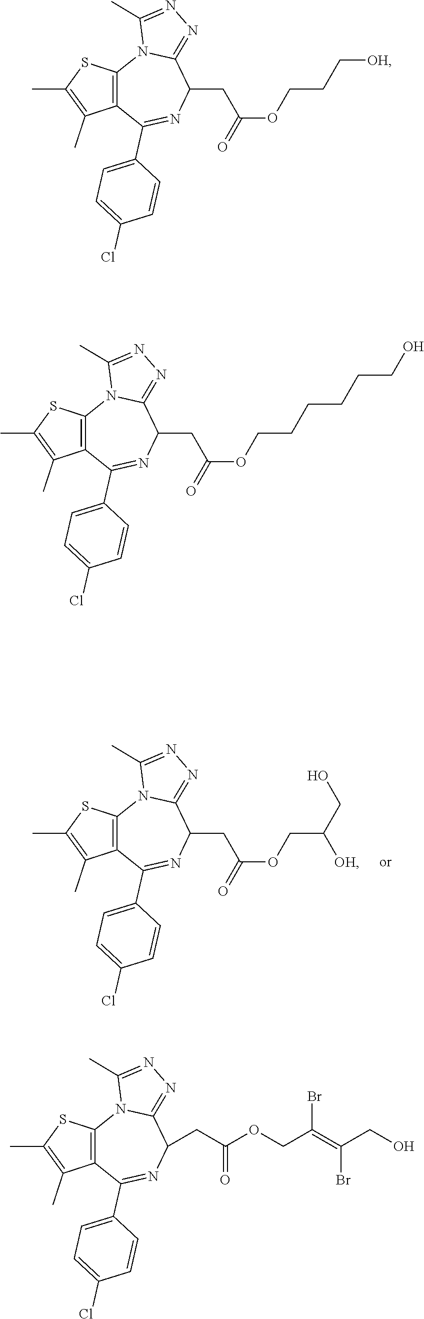 [SMILES: Cc1sc2c(c1C)C(c1ccc(Cl)cc1)=NC(CC(=O)OCCCO)c1nnc(C)n1-2 ; Cc1sc2c(c1C)C(c1ccc(Cl)cc1)=NC(CC(=O)OCCCCCCO)c1nnc(C)n1-2 ; Cc1sc2c(c1C)C(c1ccc(Cl)cc1)=NC(CC(=O)OCC(O)CO)c1nnc(C)n1-2 ; Cc1sc2c(c1C)C(c1ccc(Cl)cc1)=NC(CC(=O)OC/C(Br)=C(\Br)CO)c1nnc(C)n1-2]
or a pharmaceutically acceptable salt thereof.
